(19) United States
(12) Patent Application Publication
(10) Pub. No.: US 2016/0331952 A1
(43) Pub. Date: Nov. 17, 2016

(54) EXTERNAL PROGRAMMER

(71) Applicants: Michael A. FALTYS, Valencia, CA (US); Jacob A. LEVINE, West Hempstead, NY (US); Jesse M. SIMON, Los Angeles, CA (US); Anthony ARNOLD, Valencia, CA (US)

(72) Inventors: Michael A. FALTYS, Valencia, CA (US); Jacob A. LEVINE, West Hempstead, NY (US); Jesse M. SIMON, Los Angeles, CA (US); Anthony ARNOLD, Valencia, CA (US)

(21) Appl. No.: 15/153,639

(22) Filed: May 12, 2016

Related U.S. Application Data
(60) Provisional application No. 62/160,521, filed on May 12, 2015.
(60) Provisional application No. 62/286,952, filed on Jan. 25, 2016.

(51) Int. Cl. A61N 1/02; A61N 1/36; A61N 1/372; A61N 1/05

(52) U.S. Cl. CPC A61N 1/025; A61N 1/0556; A61N 1/36053; A61N 1/3606; A61N 1/36125; A61N 1/37247

(57) ABSTRACT

An external programmer, such as an external clock synchronization tool, can be used to update or modify the stimulation protocol and/or parameters on an implanted electrical stimulator. In particular, described herein are external clock synchronization tools that can be used to calibrate a low-power clock of the implanted electrical stimulator. These tools may also be used to provide command overrides, including suspending or delaying neurostimulation, stopping neurostimulation, and/or on-demand neurostimulation.

### CROSS REFERENCE TO RELATED APPLICATIONS

This application claims priority to U.S. Provisional Patent Application No. 62/160,521, filed May 12, 2015, and U.S. Provisional Patent Application No. 62/286,952, filed Jan. 25, 2016, each of which is incorporated by reference it its entirety for all purposes.

Some variations of the methods and apparatuses described in this patent application may be related to the following pending U.S. patent applications: U.S. patent application Ser. No. 12/620,413, filed on Nov. 17, 2009, titled “DEVICES AND METHODS FOR OPTIMIZING ELECTRODE PLACEMENT FOR ANTI-INFLAMATORY STIMULATION,” now U.S. Pat. No. 8,412,338; U.S. patent application Ser. No. 12/874,171, filed on Sep. 1, 2010, titled “PRESCRIPTION PAD FOR TREATMENT OF INFLAMMATORY DISORDERS,” Publication No. US-2011-0054569-A1; U.S. patent application Ser. No. 12/917,197, filed on Nov. 1, 2010, titled “MODULATION OF THE CHOLINERGIC ANTI-INFLAMMATORY PATHWAY TO TREAT PAIN OR ADDICTION,” Publication No. US-2011-0106208-A1; U.S. patent application Ser. No. 12/978,250, filed on Dec. 23, 2010, titled “NEURAL STIMULATION DEVICES AND SYSTEMS FOR TREATMENT OF CHRONIC INFLAMMATION,” now U.S. Pat. No. 8,612,002; U.S. patent application Ser. No. 12/797,452, filed on Jun. 9, 2010, titled “NERVE CUFF WITH POCKET FOR LEADLESS STIMULATOR,” now U.S. Pat. No. 8,886,339; U.S. patent application Ser. No. 13/467,928, filed on May 9, 2012, titled “SINGLE-PULSE ACTIVATION OF THE CHOLINERGIC ANTI-INFLAMMATORY PATHWAY TO TREAT CHRONIC INFLAMMATION,” now U.S. Pat. No. 8,788,034; and U.S. patent application Ser. No. 13/338,185, filed on Dec. 27, 2011, titled “MODULATION OF SIRTUINS BY VAGUS NERVE STIMULATION,” Publication No. US-2013-0079834-A1. Each of these patent applications is herein incorporated by reference in its entirety.

Some variations of the methods and apparatuses described in this patent application may be related to the following pending PCT application: International Application No. PCT/US2014/033690, filed Apr. 10, 2014, titled “CLOSED-LOOP VAGUS NERVE STIMULATION,” Publication No. WO 2014/169145, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Embodiments of the invention relate generally to neuromodulation of the vagus nerve for the treatment of inflammation, and more specifically to using an external programmer to effect neuromodulation of the vagus nerve.

### BACKGROUND

Neurostimulation has been used to treat a variety of diseases, including inflammatory diseases such as rheumatoid arthritis. Electrical stimulation to a nerve, such as the vagus nerve, can be delivered using, for example, an implanted stimulator. The stimulator can be patient controlled, meaning the patient controls delivery of the electrical stimulations, or the stimulator can deliver the stimulation automatically. In patient controlled systems, compliance with the prescribed dosing regimen can be inconsistent and/or poor due to the patient forgetting to deliver the stimulation, for example.

In systems where the stimulation is delivered automatically by the stimulator, the stimulation may cause some undesirable short term side effects, such as voice change or loss. This voice change or loss can be inconvenient during various activities, such as at work or during social engagements.

Therefore, in order to improve compliance with the dose schedule prescribed by the patient's physician, it would be desirable to provide the patient a reminder to deliver a therapeutic stimulation when the patient is using a patient controlled stimulator. For patients using an automated stimulator, it would be desirable to provide a warning in advance of the stimulation so that the patient can delay the stimulation if inconvenient or prepare himself for the stimulation.

In addition, it may be desirable to allow the dosing regimen and/or stimulation protocol to be adjusted, and to make delivering manual stimulation easier.

Most implantable neurostimulators include an on-board timer or oscillator that may operate as a clock and/or calendar, and may be used for timing of stimulation based on a prescribed dosing schedule. Because of this, the clocks must be sufficiently accurate so that accurate and correct dosing (stimulation) may be applied. Unfortunately, accurate clocking circuits and/or circuit elements consume a large amount of power, increasing the overall power needs of the implant. Although lower-power clocking elements are available, they are typically inaccurate, drifting by as much as 5-10 minutes per day, often unpredictably. Thus, it would be desirable to provide a low-power, and somewhat inaccurate, clocking mechanism in an implant with a mechanism that can enhance the overall accuracy of the low-power clock without substantially increasing the power used by the implant, particularly in situations where the implant may be charged less than once per week, once per month, or even once per year. Described herein are apparatuses (systems and methods) that may address the needs described herein.

### SUMMARY OF THE DISCLOSURE

The present invention relates generally to apparatuses, which may include systems or devices, including a programmable electrical vagus nerve stimulator (which may be referred to herein as a microregulator, microstimulator, neurostimluator, stimulator or vagus stimulator) that may be implanted directly against a vagus nerve so that electrical contacts on the stimulator only partially surround the circumference of the nerve Also described herein are external programmers (which may also be referred to herein as energizers) that may communicate with, provide dosing instructions to, and receive information from, the implant. The external programmer may be wearable, such as a device worn on the wrist or around the neck or attached to clothing, or may be carried in a pocket or may be integrated with a smartphone as an attachment and/or application.

For example, described herein are apparatuses (e.g., systems) for stimulating the vagus nerve of a patient that include: a programmable electrical stimulator configured to be implanted in the patient's body such that the electrical stimulator is in electrical communication with the vagus nerve, wherein the programmable electrical stimulator is configured to deliver electrical stimulation according to a first set of stimulation parameters; and an external programmer having a processor programmed to wirelessly update the first set of stimulations parameters on the programmable electrical stimulator.

The external programmer and the programmable electrical stimulator may be configured to communicate with each other through near field communication. The external programmer may be configured to be worn around the patient's wrist, e.g., as a smart watch.

The system may also include a handheld (e.g., wireless) device (e.g., smart phone, pad, etc.) that is configured to wireless communicate with the external programmer. The system may also or alternatively include a remote computer processor programmed to communicate with the smart phone via a cloud computing network. The external programmer may be configured to interface with a local computing device, e.g., wirelessly, or in some variations using a wired connection.

Also described herein are methods of using these apparatuses, including methods of updating a set of stimulation parameters in an implanted electrical stimulator. For example, a method may include: modifying the set of stimulation parameters on an external programmer; and wirelessly transmitting the modified set of stimulation parameters to the implanted electrical stimulator.

Any of these methods may also include activating a near field communication protocol, and/or determining whether the external programmer is within near field communications range of the electrical stimulator.

In any of these examples, the method may include pairing the external programmer with the electrical stimulator before the step of wirelessly transmitting the modified set of stimulation parameters.

A method of remotely modifying a set of stimulation parameters in an implanted electrical stimulator may include: modifying the set of stimulation parameters on a remote computing device; transmitting the modified set of stimulation parameters to a cloud computing network or a server; transmitting the modified set of stimulation parameters from the cloud computing network or the server to an external programmer; and wirelessly transmitting the modified set of stimulation parameters from the external programmer to the implanted electrical stimulator.

The step of transmitting the modified set of stimulation parameters from the cloud computing network or the server to the external programmer may include: transmitting the modified set of stimulation parameters from the cloud computing network or the server to a mobile device; and transmitting the modified set of stimulation parameters from the mobile device to the external programmer.

A system for stimulating the vagus nerve of a patient may include: a programmable electrical stimulator configured to be implanted in the patient's body such that the electrical stimulator is in electrical communication with the vagus nerve, wherein the programmable electrical stimulator is configured to deliver electrical stimulation according to a first set of stimulation parameters; and an external programmer having a processor programmed to provide the patient a warning or reminder a predetermined period of time before an upcoming stimulation. The external programmer may be further programmed to display to the patient a level of power remaining in the electrical stimulator. The external programmer may be further programmed to generate an alert when a level of power remaining in the electrical stimulator drops below a predetermined threshold.

Also described herein are implants (programmable electrical stimulators) that include an indelible memory for recording a log of one or more of the activity (e.g., doses delivered), instructions received, sensor information (e.g., electrical impedance of electrodes, temperature, etc.), error codes, patient overrides (“stops” or suspending of dose delivery), or any other operation parameter. For example, a system for stimulating the vagus nerve of a patient may include: a programmable electrical stimulator configured to be implanted in the patient's body, the stimulator comprising: a pair of electrodes configured to be placed in electrical communication partially around the vagus nerve; a processor configured to deliver electrical stimulation from the electrodes according to a first set of stimulation parameters; and an indelible memory configured to keep an indelible record of stimulation delivered to the vagus nerve; and an external programmer having a processor programmed to provide the patient a warning or reminder a predetermined period of time before an upcoming stimulation.

Embodiments of the present invention relate systems and methods for calibrating a first (e.g., low-accuracy) clock within an implantable device with a more accurate secondary clock, which may be external to the implantable neurostimluator. In some variations the second, more accurate clock may be incorporated into the implantable neurostimulator but kept in an ‘off’ or sleep (powered down) statue until activated, when it can synchronize the first low-power clock. The first (e.g., low-power, central) clock may be the primary time keeping mechanism within the implantable device. While not all implantable devices require a time-keeping unit, those that provide periodic outputs to the patient often require a method for keeping time that contribute to controlling when an output is given.

The low-power, low-accuracy clock in the implantable neurostimulator may be, as one non-limiting example, an internal CMOS oscillator system that acts as the clock. Low variation CMOS oscillator may be used at relatively low power, however conventional CMOS oscillators may have a relatively high degree of drift and may require correction (e.g., trimming), which is often performed manually using dedicated, external tester resources. Some CMOS oscillators use a resistive-capacitive (RC) structure.

For example, a conventional RC oscillator with a Schmitt trigger may be used as part of a low-power clock (oscillator). Such an RC oscillator may include a capacitor (C), a Schmitt trigger and a resistor (R). The output clock may have a frequency that is directly proportional to the RC product. A typical integrated poly-silicon resistor can provide a relative frequency variation of about +/−25% and a typical integrated CMOS capacitor can provide a relative frequency variation of about +/−15%. In this case, the overall relative frequency variation can have a +/−40% total frequency variation. Other RC oscillator topologies (e.g., charging and discharging an internal capacitor with controlled current, etc.) may have similar inaccuracies. Thus, such low-power oscillators can vary greatly, resulting in a high degree of variation.

In general, the low-power clocks (oscillators) described herein may vary by more than 2 minutes per day, more than 5 minutes per day, more than 10 minutes per day, more than 15 minutes per day, more than 20 minutes per day, more than 25 minutes per day, more than 30 minutes per day, more than 35 minutes per day, more than 40 minutes per day, more than 45 minutes per day, more than 50 minutes per day, or more than one hour per day (e.g., +/−2%, 3%, 4%, 5%, 6%, 7%, 8%, 9%, 10%, 11%, 12%, 13%, 14%, 15%, 16%, 17%, 18%, 19%, 20%, etc.).

For example, described herein are implantable neurostimulator system using an implantable low-power clock, the system comprising: an implantable neurostimulator having a first clock configured to keep time within the implantable neurostimulator, a rechargeable battery, a pair of electrodes configured to be placed in electrical communication partially around a nerve, and a processor configured to deliver electrical stimulation from the electrodes according to a set of stimulation parameters; and an external clock synchronizing tool, the external synchronizing tool configured comprising a second clock having more accurate time-keeping capabilities than the first clock and control circuitry configured to wirelessly communicate with the implantable neurostimulator and transmit a calibration signal, wherein the processor is further configured to calibrate the first clock based on the calibration signal.

The external clock synchronizing tool may and the implantable neurostimulator may be configured to communicate with each other through near field communication, or any other wireless communication technique, including inductive communication.

In general, the external clock synchronizing tool (which may be referred to herein as one variation of an external programmer) may be configured to be worn on the patient's neck or torso, placed near the patient while the patient sleeps (e.g., on or under a pillow, bed, or bedside, etc.), worn around the patient's wrist, etc. For example, the external clock synchronizing tool may be a smart watch, broach, necklace, pin, etc.

The external clock synchronizing tool may be rechargeable.

The external clock synchronizing tool may be configured to interface with a local computing device (computer, laptop, smartphone, etc.).

In general, as mentioned above, the first clock may be a low-power, and relatively low accuracy clock/oscillator. For example, the first clock may comprise an RC oscillator. The first clock may vary (in measuring time) by greater than 5 minutes per day (e.g., greater than 10 min per day, greater than 15 min per day, etc.).

In general, the external clock synchronizing tool may be configured to periodically transmit the calibration signal (e.g., once per second, once per minute, once per 5 min, once per 10 min, once per 15 min, once per 20 min, once per 25 min, once per 30 min, once per 35 min, once per 45 min, once per hour, etc.). Alternatively the external clock synchronizing tool may be configured to transmit continuously. Alternatively or additional, the external clock synchronizing tool may be configured to transmit when activated or triggered by a user.

In any of the variations described herein, the external clock synchronizing tool may also be configured to stop, pause or manually trigger application of neurostimulation on the nerve (e.g., vagus nerve) onto which it is implanted. For example the external clock synchronizing tool may include one or more button or other control(s) for stopping, pausing/delaying (e.g., for a selectable or predetermined time period) or manually triggering neurostimulation.

In general, communication between the external clock synchronizing tool and the implant maybe one-way (e.g., from the external clock synchronizing tool to the implant) or it may be two-way, and may include, for example, confirmation that the implant has received a synchronization signal from the external clock synchronizing tool.

Also described herein are methods of calibrating a clock within an implantable neurostimulator device. For example, a method of periodically calibrating a clock within an implantable neurostimulator device may include: keeping time in the implantable neurostimulator device using a first clock located within the implantable neurostimulator device, wherein the first clock runs continuously; wirelessly transmitting a calibration signal from an external clock synchronizing tool to the implantable neurostimulator device, wherein the external clock synchronization tool includes a second clock having more accurate time-keeping capabilities than the first clock; and calibrating the first clock in the implantable neurostimulation device based on the calibrated signal received by the implantable neurostimulator device from the external clock synchronization tool.

For example a method of calibrating a clock within an implanted neurostimulator device may include: keeping time in the implanted neurostimulator device using a first clock located within the implanted neurostimulator device, wherein the first clock runs continuously; periodically wirelessly transmitting a calibration signal from an external clock synchronizing tool to the implanted neurostimulator device, wherein the external clock synchronization tool includes a second clock having more accurate time-keeping capabilities than the first clock; and calibrating the first clock in the implanted neurostimulation device based on the calibrated signal received by the implanted neurostimulator device from the external clock synchronization tool.

As mentioned above, wirelessly transmitting a calibration signal may comprise transmitting by a near field communication protocol or any other appropriate wireless communication technique.

Any of these methods may also include applying neurostimulation to a patient's vagus nerve at a predetermined schedule based on output from the first clock. For example, neurostimulation may be delivered on a schedule (e.g., once per day, twice per day, three time per day, four time per day, etc., and/or at clock/calendar defined dates and times such as 9 am, 1 pm, 8 pm, etc.) that is kept by the first clock; the first clock may be kept accurate or within acceptable parameters based on synchronization from the external clock synchronizing tool.

In general, the external clock synchronizing tool may include a body containing a battery, wireless communication circuity, a controller, and the second (higher accuracy) clock. As already mentioned the external clock synchronizing tool may be worn on or positioned near the user in whom the implant has been implanted. For example, wirelessly transmitting a calibration signal may comprise transmitting the calibration signal when the external clock synchronizing tool is worn on a subject's neck or torso.

In general, any of the methods described herein may include transmitting an on-demand stimulation request from the external clock synchronizing tool and receiving the on-demand stimulation request in the implantable neurostimulator device, wherein receipt of the on-demand stimulation request causes the implantable neurostimulator device to deliver electrical stimulation from a pair of electrodes to a subject's vagus nerve.

Any of the methods described herein may include transmitting a hold stimulation request from the external clock synchronizing tool and receiving the hold stimulation request in the implantable neurostimulator device, wherein receipt of the hold stimulation request causes the implantable neurostimulator device to suspend electrical stimulation from a pair of electrodes to a subject's vagus nerve.

The methods of synchronizing a low-power clock of an implant described herein may also include pairing the external clock synchronizing tool with the implantable neurostimulator device before wirelessly transmitting the calibration signal.

Also described herein are implantable neurostimulator devices having low-power clock calibration systems. Such a device may include: a first clock configured to keep time within the implantable neurostimulator; a second clock having more accurate time-keeping capabilities than the first clock, wherein the second clock is in an off or idle mode while the first clocking is running; and control circuitry configured to be triggered by an event such that upon triggering, the control circuitry turns on the second clock, and uses the second clock to calibrate the the first clock, then turns the second clock back off.

The first clock may count time based upon a reference voltage generated within a circuitry of the implantable device. The second clock may comprises a piezoelectric crystal oscillator. The control circuitry may be configured to be triggered by an event comprising a preset signal programmed into the control circuitry.

In some variations, the event or trigger is thermal, e.g., temperature change.

In some variations, the preset signal may be based on a set length of time, such as a few hours, a day, a few days, a week, a couple of weeks, a month, or a few months. The preset signal may be a voltage value above a certain threshold.

Also described herein are methods of calibrating a neurostimulator. For example, a method of calibrating a clock within an implantable neurostimulator device may include: keeping time using a first clock of the implantable neurostimulator device, wherein the first clock runs continuously and is operating based upon a reference voltage generated within a circuitry of the implantable neurostimulator device; triggering a calibration protocol; turning on a reference clock within the implantable neurostimulator device; and calibrating the first clock based on the reference clock to correct for thermally-dependent time drift; and turning off the reference clock.

The first clock may comprise a reference voltage associated with an RC circuit to produce a time reference.

As mentioned above, the event that triggers the calibration may be thermal or temporal. For example, the event that triggers the calibration protocol may be a period of time (e.g., as determined by the first clock). The length of time may be a few hours, a day, a few days, a week, a couple of weeks, a month, a few months, and a year.

In some variations, the event that triggers the calibration protocol may be a change in the reference voltage above a threshold value.

As mentioned, the second clock may comprise a piezoelectric clock.

Also described herein are neurostimulator devices including these self-calibrating clocks. For example, described herein are leadless, implantable microstimulator devices for treating chronic inflammation. Such a device may include: a housing; at least two electrically conductive contacts disposed on the housing; a resonator within the sealed capsule body, the resonator comprising a coil and a capacitor configured to resonate at a predetermined frequency range; a battery within the housing; and an electronic assembly within the housing; wherein the electronic assembly comprises power management circuitry configured to receive power from the resonator to charge the battery, a microcontroller configured to control stimulation of the vagus nerve from the electrically conductive contacts, a first clock configured to keep time, a second clock having more accurate time-keeping capabilities than the first clock, wherein the second clocking is configured to periodically calibrate the first clock.

While having a central clocking module that is able to keep highly accurate time would be ideal, higher accuracy time-keeping modules are not only more expensive, but also require more power. Thus, it would be advantageous to have an internal clocking arrangement that is able to provide sufficient clocking accuracy for the lifetime of the implanted device but does not drain the power from the implanted device in an inordinately quick fashion.

Described herein are clock calibration systems contained within an implantable device. The system includes a first clocking module configured to keep time within the implantable device for the majority of the time. The system also includes a second clocking module that possesses more accurate time-keeping capabilities that only turns on when a calibration routine is triggered. For the remainder of the time, the second clocking module is either in an OFF or idle mode. The triggering event may be the passage of a certain amount of time, or by a threshold parameter being met. In some instances, the triggering event may be a preset signal programmed into the control circuitry. the preset signal is based on a set length of time, such as a few hours, a day, a few days, a week, a couple of weeks, a month, or a few months. The preset signal may also be a voltage or current value above a certain threshold value.

The system also includes control circuitry that is able to coordinate signals triggered by the event and signals sent to the central clocking module and the secondary clocking module. The system also may include a calibration module that corrects any time drifts within the first or central clocking module after the clocking calibration has been performed. In some examples, the first clocking module are able to measure and count time based upon a reference voltage generated within general circuitry of the implantable device. In some instances, the more accurate secondary time keeping module is a piezoelectric crystal oscillator.

Also disclosed herein, is a method of calibrating a central clocking module within an implantable device. The method includes obtaining a clocking value associated with the central clocking module, where the clocking value is associated with how the central clocking module keeps time, establishing an event that will trigger a calibration protocol of the clocking module using a reference clocking module, activating the reference clocking module from an OFF mode to an active mode, calibrating the central clocking module based on the reference voltage, correcting any time drifts within the central clocking module, and turning off the reference clocking module. The clocking value is associated with a reference voltage associated with a reference voltage associated with the clocking module charges an RC circuit to produce a time reference. The events that trigger the calibration step may be the running of a set amount of time where at the end of such a period of time, a calibration routine is run. The length of time may be a few hours, a day, a few days, a week, a couple of weeks, a month, a few months, and a year. The event that triggers the calibration protocol may also be a change in the reference voltage above a threshold value.

Also disclosed herein are implantable microstimulation devices for treating chronic inflammation. The implantable device may include a housing, at least two electrically conductive contacts disposed on the housing, a resonator within the sealed capsule body, where the resonator comprising a coil and a capacitor configured to resonate at a predetermined frequency range, a battery within the housing, and an electronic assembly within the housing. The electronic assembly may include a power management circuitry configured to receive power from the resonator to charge the battery, a microcontroller configured to control stimulation of the vagus nerve from the electrically conductive contacts, a first clocking module configured to keep time, a second clocking module having more accurate time-keeping capabilities than the first clocking module, and where the second clocking module is configured to periodically calibrate the first clocking module.

In some embodiments, a system for stimulating the vagus nerve of a patient is provided. The system may include an external programmer having a first clock, and a low power signal transmitter configured to generate a low power signal having a predetermined frequency. The low power signal may include time data from the first clock. The system further includes a programmable electrical stimulator configured to be implanted in the patient's body such that the electrical stimulator is in electrical communication with the vagus nerve. The programmable electrical stimulator may further include a second clock that is less accurate than the first clock, a low power signal detector tuned to the predetermined frequency, an antenna in communication with the low power signal detector for receiving the low power signal, a signal processing unit in communication with the low power signal detector and configured to process and extract the time data from the low power signal, and a controller configured to calibrate the second clock based on the time data extracted from the low powered signal.

In some embodiments, the programmable electrical stimulator further includes an amplifier configured to amplify the signal passed from the low power signal detector.

In some embodiments, the low power signal further includes instructions or commands and the signal processing unit is configured to extract the instructions or commands from the low power signal and the controller is configured to execute the instructions or commands.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A shows one variation of a system for modulating chronic inflammation including a leadless microstimulator (shown connected to the vagus nerve) and an external charger/controller.

FIG. 1B shows another variation of a system for modulating chronic inflammation, including a microstimulator, charger (“energizer”), and system programmer/controller (“prescription pad”).

FIG. 1C shows another variations of a system for modulating chronic inflammation, including a microstimulator, a securing device (POD) for securing the leadless stimulator to the nerve, an external charger, a system programmer/controller (“prescription pad”) and an optional surgical tester.

FIG. 1D is a block diagram schematically illustrating the microstimulator and the charger.

FIG. 2 illustrates one variation of an external system programmer/controller wirelessly connected to a microstimulator.

FIG. 3A shows one variation of a microstimulator in a POD configured to surround a nerve of the inflammatory reflex.

FIG. 3B shows an enlarged view of the microstimulator and POD.

FIG. 3C shows another variation of a microstimulator.

FIG. 3D shows the microstimulator of FIG. 3C within a POD.

FIG. 3E shows another variation of the microstimulator.

FIG. 4 shows a schematic diagram of a microstimulator and POD around vagus nerve.

FIG. 5 is a flowchart that illustrates one embodiment of a reminder system.

FIG. 6 is a flowchart that illustrates another embodiment of a reminder system.

FIG. 7A is a schematic of an embodiment of an external programmer.

FIG. 7B is an illustration of an embodiment of system for remotely modifying the stimulation parameters of an implanted stimulator using an external programmer.

FIG. 8 is a flow chart that illustrates one embodiment of updating the stimulation protocol on an electrical stimulator using an external programmer.

FIG. 9 is a flow chart that shows an embodiment of pairing an external programmer with an electrical stimulator.

FIG. 10 is a flow chart that shows an embodiment of updating the stimulation protocol on an electrical stimulator using a local computing device and an external programmer.

FIG. 11 is a flow chart that shows an embodiment of updating the stimulation protocol on an electrical stimulator using a remote computing device and an external programmer.

FIG. 12 is a flow chart that shows how an external programmer can by synchronized with an electrical stimulator so that reminders or warnings can be provided to the patient.

FIG. 13 is a flow chart that shows how an external programmer can be used to deliver manual stimulation and monitor patient compliance.

FIG. 14 shows one variation of an implant as described herein, including an indelible memory for logging activity of the implant and/or programmer.

FIG. 15 is a flowchart showing the steps of calibrating a first clocking module (“first clock”) with a second clocking module (“second clock”).

FIG. 16 is a diagram showing calibration of a first clocking module by a second clocking module based on a change in voltage.

FIG. 17 is a diagram showing calibration of a first clocking module by a second clocking module based on a pre-determined period of time.

FIG. 18 is a schematic showing an embodiment of a stimulator with a low power signal detector circuit.

FIG. 19 is a schematic showing an embodiment of an external programmer with a low power signal transmitter.

### DETAILED DESCRIPTION

In general, described herein are methods and apparatuses for performing these methods, of applying a communication to a patient from an implanted therapeutic device. This communication may be referred to as a notice, warning, alert, reminder, or prompt, and typically includes a stimulation that is detectable by the patient, but is distinct from a therapeutic dose from the implant. The methods and apparatuses described herein, including the example, are directed primarily to vagus nerve stimulation apparatuses and methods; however, it should be understood that these methods and apparatuses may be used with virtually any implanted therapeutic stimulation device, including microstimulators that are not connected to the vagus nerve.

In general, the communication may be referred to herein as a prompt, notice, warning, alert, reminder, or the like, which may be used interchangeable and/or may refer to the goal or function of the communication; they may otherwise have similar or identical characteristics. For example, a prompt, notice, warning, alert, or reminder (referred to for convenience as a prompt) may be patient-detectable stimulation that is of the same mode (e.g., electrical, mechanical, etc.) as the therapeutic stimulation (dose) provided by the implant. In some variations, but not all, the prompt has stimulation parameters that are sub-therapeutic compared to a therapeutic dose. For example, the prompt may have stimulation parameters that are lower in intensity (e.g., current amplitude, voltage, etc.), frequency, and/or duration than the therapeutic dose being delivered to that patient. In some variations, the prompt stimulation range (also referred to below as “reminder stimulation range”) may be within the same range limits as therapeutic doses (e.g., between 1-5000 μA, etc.). The prompt stimulation may be adjusted separately (by a user, clinician, technician, etc.), so that it is noticeable by the patient (whereas in some variations the dose stimulation may not be immediately noticeable). For example, for a particular patient with an implanted therapy device, a dose may be set at an effective stimulation dose (amplitude, frequency, duration, etc.), and the prompting dose may be selected so that it is distinct from the stimulation dose and detectable by the patient. In some variations the prompt stimulation may have a greater amplitude (e.g. current amplitude) but may have a shorter duration and/or different frequency.

### Vagus Nerve Stimulation System

Systems for electrically stimulating one or more nerves to treat chronic inflammation may include an implantable, wireless microstimulator such as those described herein and an external charging device (which may be referred to as a charging wand, charger, or energizer). In some variations the system also includes a controller such as a “prescription pad” that helps control and regulate the dose delivered by the system. The microstimulator may be secured in position using a securing device (which may be referred to as a “POD”) to hold the microstimulator in position around or adjacent to a nerve. These microstimulators are designed and adapted for treatment of chronic inflammation, and may be configured specifically for such use. Thus, an implantable microstimulator may be small, and adapted for the low duty-cycle stimulation to modulate inflammation. For example, the implantable microstimulator may hold a relatively small amount of power over weeks or even months and discharge it at a rate sufficient to modulate the anti-inflammatory pathway without significantly depressing heart rate or triggering any number of unwanted effects from the vagus nerve or other neural connections. Any of the nerves of the inflammatory reflex, including the vagus nerve, may be treated as described herein using the systems described.

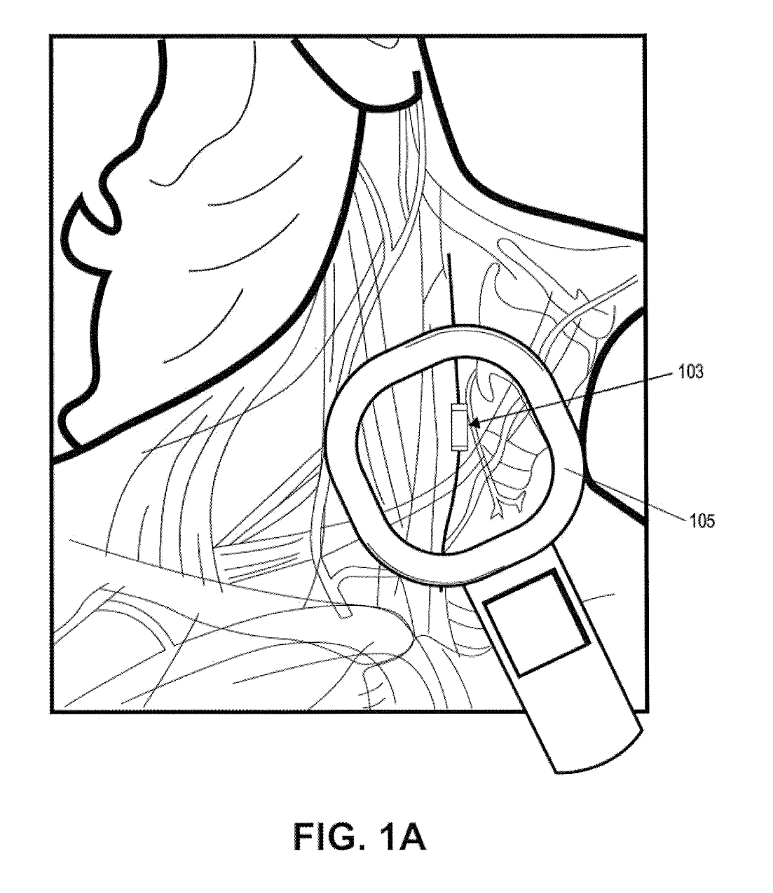

For example, FIG. 1A illustrates one variation of a system for treating chronic inflammation that includes a microstimulator contained in POD that is mounted on cervical vagus nerve and charged a programmed by an external charger/programmer unit. This variation of a system includes a microstimulator 103 that has been implanted to contact the vagus nerve as shown. The implant may be programmed, controlled and/or charged by a charger/controller 105 device. In this variation the charger/controller is a loop with a wand region.

FIG. 1B shows another variation of a system for treating chronic inflammation that also includes an implantable microstimulator 103 (shown inserted into a POD to hold it in position relative to a nerve) and a charging device (“energizer” 105) configured as a collar to be worn around the subject's neck and charge the implant. Optionally, the system may include a prescription pad 107 which may be a separate dedicated device or part of a mobile or other handheld device (e.g., an application to run on a handheld device).

FIG. 1C shows another variation of a system for treating chronic inflammation. The systems described herein may also be referred to as systems for the neural stimulation of the cholinergic anti-inflammatory pathway (NCAP). These systems may be configured as chronic implantable systems. In some variations, the systems are configured to treat acutely (e.g., acute may 8 hours or less), sub-acutely (expected to occur for fewer than 30 days), or chronically (expected to occur for more than 30 days).

In general, the systems described herein may be configured to apply electrical stimulation at a minimum level necessary to modulate the inflammatory reflex (e.g., modulating cytokine release) characterized by the Chronaxie and rheobase. Chronaxie typically refers to the minimum time over which an electric current double the strength of the rheobase needs to be applied in order to stimulate the neuron. Rheobase is the minimal electrical current of infinite duration that results in an action potential. As used herein, cytokines refer to a category of signaling proteins and glycoproteins that, like hormones and neurotransmitters, are used extensively in cellular communication.

The NCAP Systems described herein are typically intended for the treatment of chronic inflammation through the use of implanted neural stimulation devices (microstimulators) to affect the Neural Stimulation of the Cholinergic Anti-inflammatory Pathway (NCAP) as a potential therapeutic intervention for rheumatologic and other inflammation-mediated diseases and disorders. Neurostimulation of the Cholinergic Anti-inflammatory Pathway (NCAP) has been shown to modulate inflammation. Thus, the treatment and management of symptoms manifested from the onset of disease (e.g., inflammatory disease) is based upon the concept of modulating the Cholinergic Anti-inflammatory Pathway. The NCAP pathway normally maintains precise restraint of the circulating immune cells. As used herein, the CAP is a reflex that utilizes cholinergic nerve signals traveling via the Vagus nerve between the brain, chemoreceptors, and the reticuloendothelial system (e.g., spleen, liver). Local release of pro-inflammatory cytokines (e.g., tumor necrosis factor or TNF) from resident immune cells is inhibited by the efferent, or indirectly by afferent vagus nerve signals. NCAP causes important changes in the function and microenvironment of the spleen, liver and other reticuloendothelial organs. Leukocytes which circulate systemically become “educated” as they traverse the liver and spleen are thereby functionally down regulated by the affected environment of the reticuloendothelial system. This effect can potentially occur even in the absence of an inflammatory condition.

Under this model, remote inflammation is then dampened by down-regulated cytokine levels. Stimulation of the vagus nerve with a specific regiment of electrical pulses regulates production of pro-inflammatory cytokines. In-turn, the down regulation of these cytokines may reduce localized inflammation in joints and other organs of patients with autoimmune and inflammatory disorders.

The NCAP System includes a neurostimulator that may trigger the CAP by stimulating the cervical vagus nerve. The NCAP System issues a timed burst of current controlled pulses with sufficient amplitude to trigger the CAP at a particular interval. These two parameters, Dose Amplitude and Dose Interval, may be used by a clinician to adjust the device. For example, the clinician may set the Dose Amplitude by modifying the current level. The Dose Interval may be set by changing the duration between Doses (e.g. 12, 24, 48 hours).

In some variations, dose amplitude may be set to within the Therapy Window. The Therapy window is defined as the lower limit of current necessary to trigger the CAP, and the upper limit is the level at which the Patient feels uncomfortable. The lower limit is called the Threshold (T), and the uncomfortable level is called Upper Comfort Level (UCL).

Dose Amplitude thresholds are nonlinearly dependent upon Current (I), Pulse width (PW), Pulse Frequency (PF), and Burst Duration (BD). Amplitude is primarily set by charge (Q), that is Current (I)×Pulse width (PW). In neurostimulation applications current has the most linear relationship when determining thresholds and working within the therapy window. Therefore, the clinician may modify Dose Amplitude by modifying current. The other parameters are held to experimentally determined defaults. Pulse width is selected to be narrow enough to minimize muscle recruitment and wide enough to be well above the chronaxie of the targeted neurons. Stimulus duration and pulse frequency was determined experimentally in Preclinical work.

Dose Interval may be specific for particular diseases and the intensity of diseases experienced by a patient. Our initial research has indicated that the cervical portion of the vagus nerve may be an ideal anatomic location for delivery of stimulation. The nerve runs through the carotid sheath parallel to the internal jugular vein and carotid artery. At this location, excitation thresholds for the vagus are low, and the nerve is surgically accessible. We have not found any significant difference in biomarker modulation (e.g., modulation of cytokines) between right and left. Even though the right vagus is thought to have lower thresholds than the left in triggering cardiac dysrythmias, the thresholds necessary for NCAP are much lower than those expected to cause such dysrythmias. Therefore a device delivering NCAP can safely be applied to either the right or left vagus.

We have also found, surprisingly, that the Therapy Window is maximized on the cervical vagus through the use of a bipolar cuff electrode design. Key parameters of the cuff may be: spacing and shielding of the contacts. For example, the contact points or bands may be spaced 1-2 diameters of the vagus nerve apart, and it may be helpful to shield current from these contacts from other nearby structures susceptible to inadvertent triggering. The cuff may be further optimized by using bands which are as long and wide as possible to reduce neurostimulator power requirements.

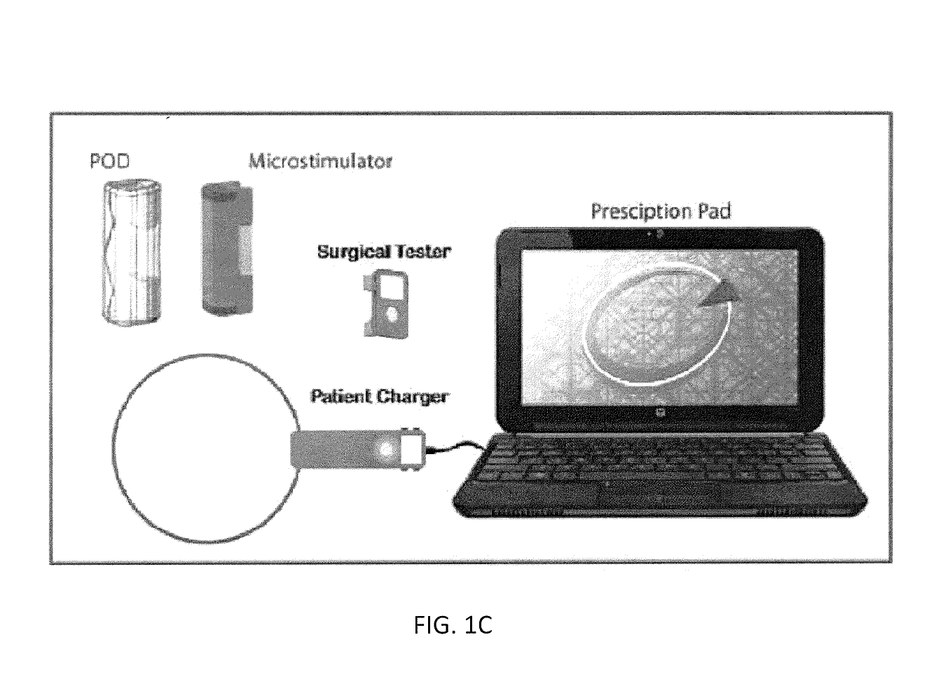

Thus, any variations of the systems described herein (e.g., the NCAP system) may be implemented with a Cuff, Lead and Implantable Pulse Generation (IPG), or a Leadless Cuff. The preferred implementation is a leadless cuff implemented by a microstimulator with integral electrode contacts in intimate contact with the nerve and contained within a Position (or protection) and Orientation Device (POD). This is illustrated in FIGS. 3A and 3B. The POD 301 may form a current shield, hold the microstimulator into place against the vagus nerve, and extend the microstimulator integral contacts with integral contacts in the POD itself. The POD is typically a polymer shell that encapsulates a microstimulator implant and that allows a nerve to run through the interior against the shell wall parallel to the length of the microstimulator implant. Within the shell of the POD, the microstimulator implant remains fixed against the Vagus nerve so the electrodes remain in contact with the nerve. The POD anchors the implant in place and prevents the implant from rotating or separating from the nerve, as well as maintaining contact between the electrodes and the nerve and preserving the orientation as necessary for efficient external charging of the microstimulator battery.

Referring back to FIG. 1C, the system may include an implantable microstimulator contained in a POD, a Patient Charger, and a prescription pad that may be used by the clinician to set dosage parameters for the patient. This system may evaluate the efficacy, safety, and usability of an NCAP technology for chronic treatment of clinical patients. The system can employ a Prescription Pad (external controller) that may include the range of treatment options.

As described in more detail in U.S. patent application Ser. No. 12/874,171, filed on Sep. 1, 2010, titled “PRESCRIPTION PAD FOR TREATMENT OF INFLAMMATORY DISORDERS,” Publication No. US-2011-0054569-A1, previously incorporated by reference in its entirety, the Prescription Pad may incorporate workflows in a simplified interface and provide data collection facilities that can be transferred to an external database utilizing commercially robust and compliant methods and procedures. In use, the system may be recommended for use by a clinician after assessing a patient; the clinician may determine that treatment of chronic inflammation is warranted. The clinician may then refer the patient to an interventional doctor to implant the microstimulator. Thereafter then clinician (or another clinician) may monitor the patient and adjust the device via a wireless programmer (e.g. prescription pad). The clinician may be trained in the diagnosis and treatment procedures for autoimmune and inflammatory disorders; the interventional placement of the system may be performed by a surgeon trained in the implantation of active neurostimulation devices, with a sufficient depth of knowledge and experience regarding cervical and vagal anatomy, experienced in performing surgical dissections in and around the carotid sheath.

The system may output signals, including diagnostics, historical treatment schedules, or the like. The clinician may adjust the device during flares and/or during routine visits. Examples of implantation of the microstimulator were provided in U.S. patent application Ser. No. 12/874,171, filed on Sep. 1, 2010, titled “PRESCRIPTION PAD FOR TREATMENT OF INFLAMMATORY DISORDERS,” Publication No. US-2011-0054569-A1. For example, the implant may be inserted by making an incision in the skin (e.g., cm) along Lange's crease between the Facial Vein and the Omohyoid muscle, reflecting the Sternocleidomastoid and gaining access to the carotid sheath. The IJV may be displaced, and the vagus may be dissected from the carotid wall (<2 cm). A sizing tool may be used to measure the vagus, and an appropriate Microstimulator and POD Kit (small, medium, large) may be selected. The POD may then be inserted under nerve with the POD opening facing the surgeon, so that the microstimulator can be inserted inside POD so that the microstimulator contacts capture the vagus. The POD may then be sutured shut. In some variations a Surgical Tester may be used to activate the microstimulator and perform system integrity and impedance checks, and shut the microstimulator off, during or after the implantation. In other variations the surgical tester may be unnecessary, as described in greater detail below.

A physician may use the Patient Charger to activate the microstimulator, perform integrity checks, and assure sufficient battery reserve exists. Electrodes may be conditioned with sub-threshold current and impedances may be measured. A Physician may charge the microstimulator. In some variations a separate charger (e.g., an “energizer”) may be used by the patient directly, separate from the controller the physician may use. Alternatively, the patient controller may include controls for operation by a physician; the system may lock out non-physicians (e.g., those not having a key, code, or other security pass) from operating or modifying the controls.

In general, a physician may establish safe dosage levels. The physician may slowly increment current level to establish a maximum limit (Upper Comfort Limit). This current level may be used to set the Dosage Level. The exact procedure may be determined during this clinical phase.

The Physician may also specify dosing parameters that specify dosage levels and dosage intervals. The device may contain several concurrent dosing programs which may be used to acclimate the patient to stimulus, gradually increase dosage until efficacy is achieved, reset tachyphylaxis, or deal with unique patient situations.

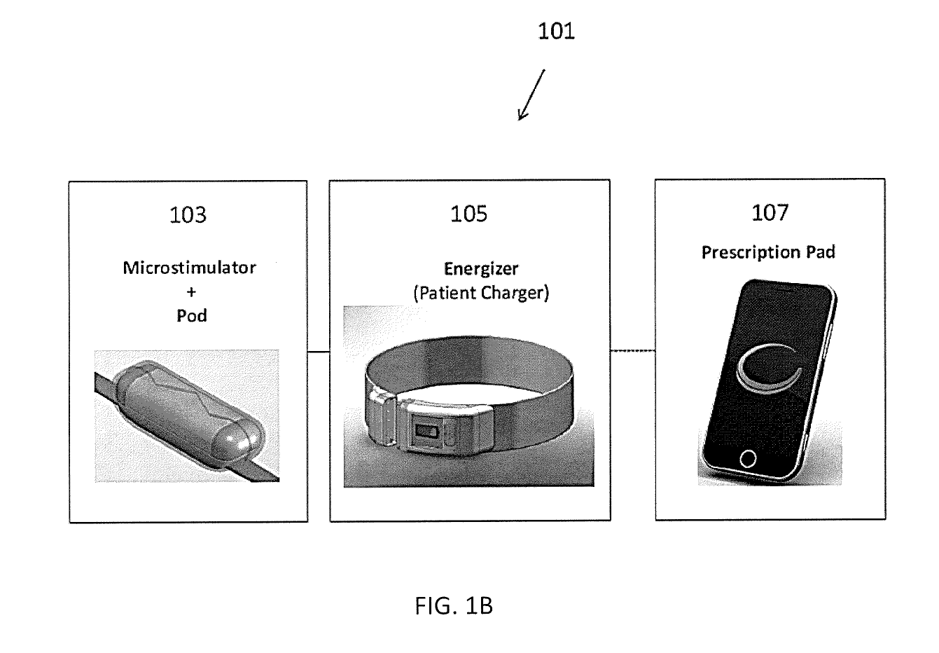

In some variations, the Prescription Pad may be configured to handle multiple patients and may index their data by the microstimulator Serial Number. For example, a Prescription Pad may handle up to 100,000 patients and 10,000 records per patient, and may store the data in its local memory and may be backed up on an external database. In some variations, during each charging session, accumulated even log contents will be uploaded to the Patient Charger for later transfer to Prescription Pad. The data may or may not be cleared from the microstimulator. For example, FIG. 2 shows the addition of a prescription pad 203 wirelessly connected to the charger/programmer 207.

The microstimulators described herein are configured for implantation and stimulation of the cholinergic anti-inflammatory pathway, and especially the vagus nerve. In particular the microstimulators described herein are configured for implantation in the cervical region of the vagus nerve to provide extremely low duty-cycle stimulation sufficient to modulate inflammation. These microstimulators may be adapted for this purpose by including one or more of the following characteristics, which are described in greater detail herein: the conductive capsule ends of the microstimulator may be routed to separate electrodes; the conductive capsule ends may be made from resistive titanium alloy to reduce magnetic field absorption; the electrodes may be positioned in a polymer saddle; the device includes a suspension (e.g., components may be suspended by metal clips) to safeguard the electronics from mechanical forces and shock; the device may include an H-bridge current source with capacitor isolation on both leads; the device may include a built in temperature sensor that stops energy absorption from any RF source by detuning the resonator; the device may include a built-in overvoltage sensor to stop energy absorption from any RF source by detuning resonator; the system may include DACs that are used to calibrate silicon for battery charging and protection; the system may include DACs that are used to calibrate silicon for precision timing rather than relying on crystal oscillator; the system may include a load stabilizer that maintains constant load so that inductive system can communicate efficiently; the system may include current limiters to prevent a current rush so that the microstimulator will power up smoothly from resonator power source; the system may extract a clock from carrier OR from internal clock; the device may use an ultra-low power accurate RC oscillator that uses stable temperature in body, DAC calibration, and clock adjustment during charging process; the device may use a solid state LIPON battery that allows fast recharge, supports many cycles, cannot explode, and is easy to charge with constant voltage; and the device may include a resonator that uses low frequency material designed not to absorb energy by high frequency sources such as MRI and Diathermy devices.

Many of these improvements permit the device to have an extremely small footprint and power consumption, while still effectively modulating the vagus nerve.

FIG. 3A is a perspective drawing of the Pod containing the microstimulator. Sutures (not shown) are intended to be bridged across one to three sets of holes. Electrodes integrated into the pod are not shown but would extend as bands originating and ending on the two outer pairs of suture holes.

In some variations, including those described above, the microstimulator consists of a ceramic body with hermetically sealed titanium-niobium ends and integral platinum-iridium electrodes attached. The microstimulator may be designed to fit within a POD 309, as shown in FIGS. 3A-3D. As described above, the POD is a biocompatible polymer with integrated electrodes that may help the microstimulator to function as a leadless cuff electrode. In some variations, such as the variation shown in FIG. 3E, contained within the hermetic space of the microstimulator 301 is an electronic assembly that contains a rechargeable battery 321, solenoid antenna 323, hybrid circuit 325 and electrode contacts (Ti Alloy braze ring and end cap) 327 at each end to make contact with the titanium/platinum case ends.

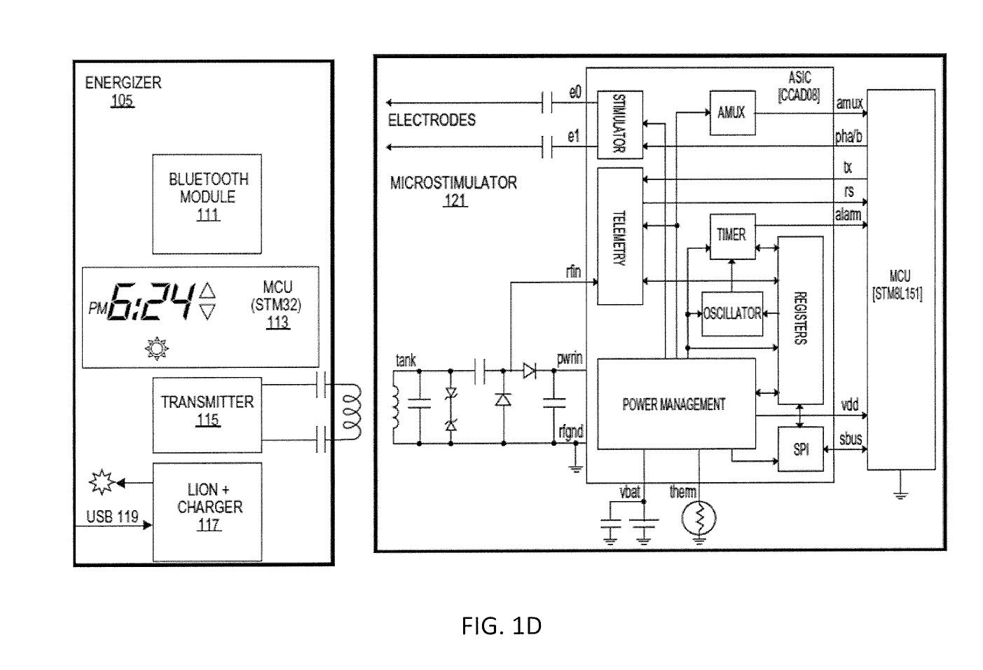

As mentioned above, some of the device variations described herein may be used with a POD to secure the implant (e.g., the leadless/wireless microstimulator implant) in position within the cervical region of the vagus nerve so that the device may be programmed and recharged by the charger/programmer (e.g., “energizer”). For example, FIG. 4 shows a schematic diagram of a POD containing a microstimulator. The cross section in FIG. 4 shows the ceramic tube containing electronic assembly that includes the hybrid, battery and coil. The rigid or semi-rigid contacts are mounted on the tube and surround the oval vagus nerve. The POD surrounds the entire device and includes a metal conductor that makes electrical contact with the microstimulator contacts and electrically surrounds the nerve.

In some variations, the microstimulator may have a bipolar stimulation current source that produce as stimulation dose with the characteristics shown in table 1, below. In some variation, the system may be configured to allow adjustment of the “Advanced Parameters” listed below; in some variations the parameters may be configured so that they are predetermined or pre-set. In some variations, the Advanced Parameters are not adjustable (or shown) to the clinician. All narameters listed in Table 1 are ±5% unless specified otherwise.

TABLE 1
Microstimulator parameters

| Property | Value | Default |
|---|---|---|
| Dosage | 0-5,000 μA in 25 μA steps | 0 |
| Amplitude | | |
| (DA) | | |
| Intervals | Minute, Hour, Day, Week, | Day |
| | Month | |
| Number of | N = 60 Maximum | 1 |
| Doses per | | |
| Interval | | |

Advanced Parameters

| | | |
|---|---|---|
| Pulse width | 50-1,000 μS in 50 μS | 200 |
| Range (PW) | increments | |
| Stimulus | 0.5-1000 seconds per | 60 |
| Duration (SD) | dose | |
| Pulse | 1-50 Hz | 10 |
| Frequency | | |
| (PF) | | |
| Stimulus | ±3.3 or ±5.5 ±1 Volts | Automatically set |
| Voltage (SV) | | by software |
| Constant | ±15% over supported range | |
| Current | of load impedances (200-2000 Ω) | |
| Output | | |
| Specific Dose | Set a specific time between | Driven by default |
| Time | 12:00 am-12:00 am in one | table (TBD) |
| | minute increments for each | |
| | Dose Issue | |
| Number of | 4 maximum | 1 |
| Sequential | | |
| Dosing | | |
| Programs | | |

The Dosage Interval is defined as the time between Stimulation Doses. In some variations, to support more advanced dosing scenarios, up to four ‘programs’ can run sequentially. Each program has a start date and time and will run until the next program starts. Dosing may be suspended while the Prescription Pad is in Programming Mode. Dosing may typically continue as normal while charging. Programs may be loaded into one of four available slots and can be tested before they start running. Low, Typical, and High Dose schedules may be provided. A continuous application schedule may be available by charging every day, or at some other predetermined charging interval. For example, Table 2 illustrates exemplary properties for low, typical and high dose charging intervals:

TABLE 2
low typical and high dose charging intervals

| Property | Value |
|---|---|
| Low Dose Days | 30 days max: 250 μA, 200 μS, 60 s, 24 hr, |
| Charge Interval | 10 Hz, ±3.3 V |
| Typical Dose | 30 days max: 1,000 μA, 200 μS, 120 s, 24 hr, |
| Charge Interval | 10 Hz, ±3.3 V |
| High Dose Charge | 3.5 days max: 5,000 μA, 500 μS, 240 s, |
| Interval | 24 hr, 20 Hz, ±5.5 V, |

The system may also be configured to limit the leakage and maximum and minimum charge densities, to protect the patient, as shown in Table 3:

TABLE 3
safety parameters

| Property | Value | |
|---|---|---|
| Hardware DC Leakage | <50 | nA |
| Protection | | |
| Maximum Charge | 30 | μC/cm²/phase |
| Density | | |
| Maximum Current | 30 | mA/cm² |
| Density | | |

In some variations, the system may also be configured to allow the following functions (listed in Table 4 below):

TABLE 4
Additional functions of the microstimulator and/or controller(s)

| Function | Details |
|---|---|
| Charging | Replenish Battery |
| Battery Check | Determine charge level |
| System Check | Self Diagnostics |
| Relative | Temperature difference from |
| Temperature | baseline |
| Program | Read/Write/Modify a dosage |
| Management | parameter programs |
| Program | Transfer entire dosage parameter |
| Up/Download | programs |
| Electrode | Bipolar Impedance (Complex) |
| Impedances | |
| Signal Strength | Strength of the charging signal to |
| | assist the patient in aligning the |
| | external Charge to the implanted |
| | Microstimulator. |
| Patient Parameters | Patient Information |
| Patient History | Limited programming and |
| | exception data |
| Implant Time/Zone | GMT + Time zone, 1 minute |
| | resolution, updated by Charger |
| | each charge session |
| Firmware Reload | Boot loader allows complete |
| | firmware reload |
| Emergency Stop | Disable dosing programs and |
| | complete power down system until |
| | Prescription Pad connected or |
| | otherwise turned off |

Stimulation Reminder and/or Warning Systems and Methods

In some embodiments, the therapeutic stimulation delivered by the microstimulator can be delivered manually by the patient according to a predetermined schedule, such as a stimulation every 4, 6, 8, 12, 24, or 48 hrs. In order to improve patient compliance to the stimulation delivery schedule, a patient detectable electrical stimulation, i.e. a reminder stimulation, can be automatically delivered by the microstimulator to remind or prompt a patient to apply a manual therapeutic stimulation. The patient detectable electrical stimulation can be delivered before the scheduled therapeutic stimulation by a predetermined amount of time, such as 0 seconds to 60 minutes before the scheduled therapeutic stimulation. In some embodiments, the patient detectable electrical stimulation can have an intensity or amplitude that is large enough to be detectable but is substantially less than the therapeutic stimulation. For example, the patient detectable electrical stimulation can have an amplitude that is less than the maximum stimulation below the pain or discomfort threshold stimulation (less than or equal to 0.5 mA, e.g., 0.6 mA, 0.7 mA, 0.8 mA, 0.9 mA, etc.), and a duration of about 10 seconds or less (e.g., 9 seconds, 8 seconds, 7 seconds, 6 seconds, 5 seconds, etc.). The frequency of the stimulation may be between 0.1 Hz and 1000 Hz (e.g., between 1-100 Hz, etc.).

In some variations the patient-detectable notification stimulation may be delivered using parameters that are distinct from the therapeutic stimulation parameters. For example, if, as described above, the therapeutic stimulation parameters are electrical stimulation of the vagus nerve at between about 1.0 to 5 mA, at between about 2-90 Hz (e.g., 10 Hz) for a dose period of between 30 sec and 400 sec, then the notification stimulation may be outside of any of these ranges but the same modality as the therapeutic stimulation (e.g., electrical stimulation of the vagus nerve at less than about 1.0 mA, between about 2-90 Hz, for less than 30 sec; electrical stimulation of the vagus nerve at less than about 1.0 mA, between about 2-90 Hz, for between about 1 sec and 2 min, etc.). In some variations the notification stimulation may be pulsed with these parameters at an envelope that is between about 0.01 to 2 Hz for a duration of between about 1 sec and 1 min, where the notification stimulation is applying current at the non-therapeutic range for an on time followed by an off-time, where the frequency of on-time and/or off-time is at the envelope frequency (e.g., 0.01 to 2 Hz).

In some embodiments, the reminder stimulation can be periodically resent after the scheduled therapeutic stimulation time has elapsed if the patient has not delivered the therapeutic stimulation by the scheduled time. For example, an additional reminder stimulation can be sent every 5 to 60 minutes until the patient delivers the therapeutic stimulation. In some embodiments, the patient can elect to delay, postpone or cancel the scheduled therapeutic stimulation. In some embodiments, the reminder stimulation intensity, such as the stimulation amplitude, can be increased by a predetermined amount, such as by 0.1, 0.2, or 0.3 mA up to a predetermined maximum, with each successive reminder when the patient fails to deliver the therapeutic stimulation.

In some embodiments, the reminders can be implemented in the microstimulator hardware and/or software, such as via a program stored in memory on the microstimulator. In some embodiments, the option to delay, postpone or cancel the therapeutic stimulation can be implemented on the charger, the prescription pad, or another external control device, such as a smartphone with an application for controlling the microstimulator.

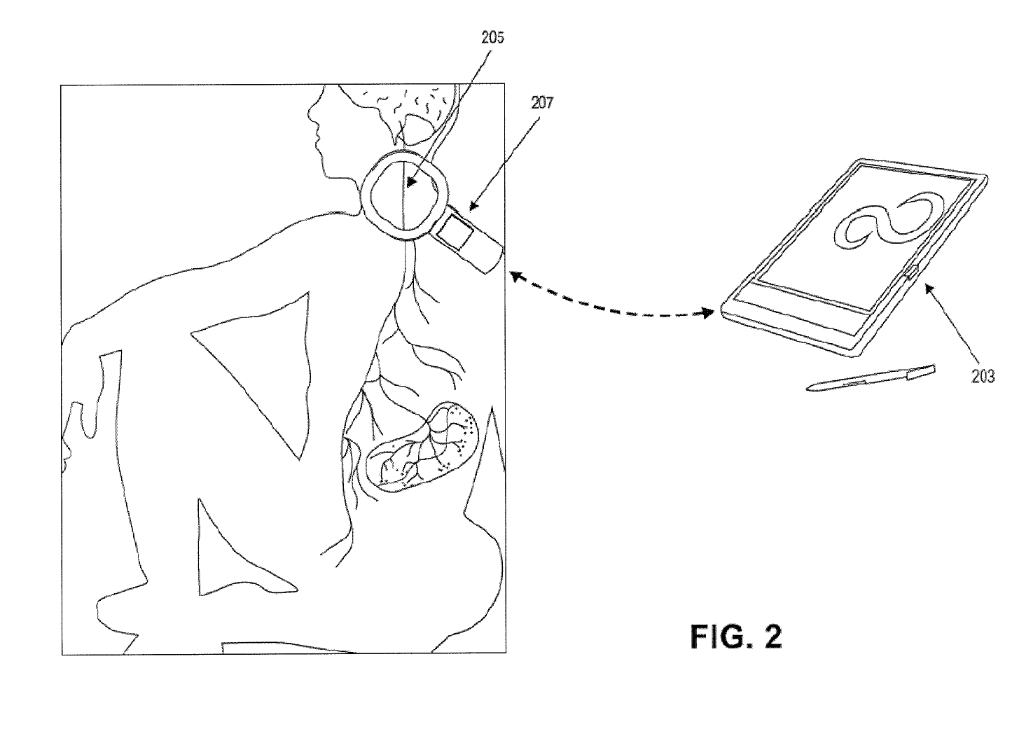

FIG. 5 is a flowchart that illustrates one embodiment of a system and method of implementing a reminder system. The reminder system can be used to provide a patient with a reminder to deliver a therapeutic stimulation using a patient controlled stimulator. The patient controlled stimulator can be a microstimulator that delivers electrical stimulation to a nerve such as the vagus nerve. In step 500, the reminder system and method can compare the current time to a programmed treatment schedule. Based on this comparison, in step 502, the system and method can determine whether the current time is within the reminder period. The reminder period can be a predetermined period of time before a scheduled therapeutic stimulation. In some embodiments, the predetermined period of time can be about 1 to 60 minutes, or about 5 to 30 minutes, or less than or equal to about 5, 10, 15, 20, 25, and 30 minutes before the schedule stimulation. In some embodiments the predetermined period of time can be adjusted by the patient and/or health care provider.

If the system and method determines that the current time is not within the reminder period, the system and method loops back again to step 500 and continues to compare the current time to the treatment schedule. In some embodiments, the system and method can perform the comparison every minute, or every 5 minutes, every 10 minutes, or at some other interval of time. If the system and method determines that the current time is within the reminder period, then it proceeds to step 504 to generate a patient detectable stimulation to remind the patient to deliver the therapeutic stimulation.

Next, in step 506, the system and method waits a predetermined period of time and in step 508 checks whether the patient has delivered the therapeutic stimulation during this predetermined period of time. If the system and method determines that the patient has not delivered the therapeutic stimulation, it loops back to step 504 to generate another patient detectable stimulation to remind the patient again to deliver the therapeutic stimulation. If the system and method determines that the patient has delivered the therapeutic stimulation, it loops back to step 500 to compare the current time to the treatment schedule to determine whether the current time is within the next reminder period.

In some embodiments, such as a VNS system that automatically delivers the VNS according to a predetermined schedule or in accordance with a stimulation algorithm, the stimulator can deliver a warning stimulation before the therapeutic stimulation is delivered automatically by the stimulator. A warning stimulation is useful in this situation because electrical stimulation of the vagus nerve can result in various side effects, such as affecting the ability to speak, altering the patient's voice, and the like. For example, providing the warning allows the patient to excuse himself or delay the scheduled stimulation if those side effects would be inconvenient at the time.

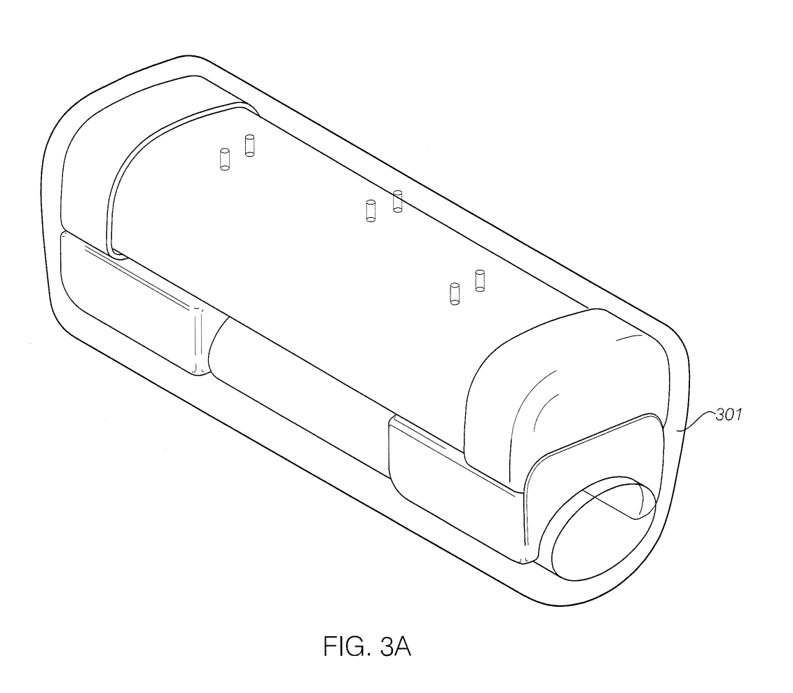

FIG. 6 is a flowchart that illustrates one embodiment of a system and method of implementing a warning system. In steps 600 and 602, the current time is compared to the treatment schedule to determine whether the current time is within a warning period preceding a scheduled therapeutic stimulation. If in step 602, it is determined that the current time is not within the warning period, the system and method loops back to step 600 to continue to compare the current time to the treatment schedule. If in step 602, it is determined that the current time is within the warning period, the system and method proceeds to step 604 to generate a patient detectable stimulation to warn the patient that therapeutic stimulation will be delivered within a predetermined period of time. The warning period can be a predetermined period of time before a scheduled therapeutic stimulation. In some embodiments, the predetermined period of time can be about 1 to 60 minutes, or about 5 to 30 minutes, or less than or equal to about 5, 10, 15, 20, 25, and 30 minutes before the schedule stimulation. In some embodiments, an additional warning period just before the therapeutic stimulation can be provided to alert the patient that a stimulation is imminent. This imminent warning period can be within 1 to 30 seconds before the stimulation is delivered. In some embodiments the predetermined period of time can be adjusted by the patient and/or health care provider.

Next, in step 606, the system and method determines whether the patient has elected to delay therapeutic stimulation. The patient can delay stimulation by, for example, selecting and/or imputing a command in a control device, such as a neck charger, a prescription pad, a mobile device, and the like that can communicate with the stimulation device. If the patient elects to delay the delivery of the therapeutic stimulation, then in step 610 the delivery of the therapeutic stimulation can be delayed by a predetermined amount of time. In some embodiments, the predetermined period of time can be about 1 to 120 minutes, or about 5 to 60 minutes. In some embodiments the predetermined period of time can be adjusted by the patient and/or health care provider. If the patient does not elect to delay the delivery of the therapeutic stimulation, then in step 608 the therapeutic stimulation is delivered to the patient and the system and method loops back to step 600 to again compare the current time to the treatment schedule to determine whether the current time is within the next warning period.

Although the patient detectable stimulations have been described as electrical stimulations, in some embodiments the patient detectable stimulation can be or include other types of stimuli, such as an audible sound, a vibration, a text message, an email, and/or a visual indicator. These other or addition stimuli can be implemented in some cases in the neurostimulator and in other cases on other devices such as a prescription pad, an external device on, for example, the wrist, a smartphone, and/or the charger. The alternative stimuli can be used instead of the electrical stimulation, or can be used in conjunction with the electrical stimulation, and can be used in any combination, which can be selected and modified by the patient and/or health care provider.

### External Programmer

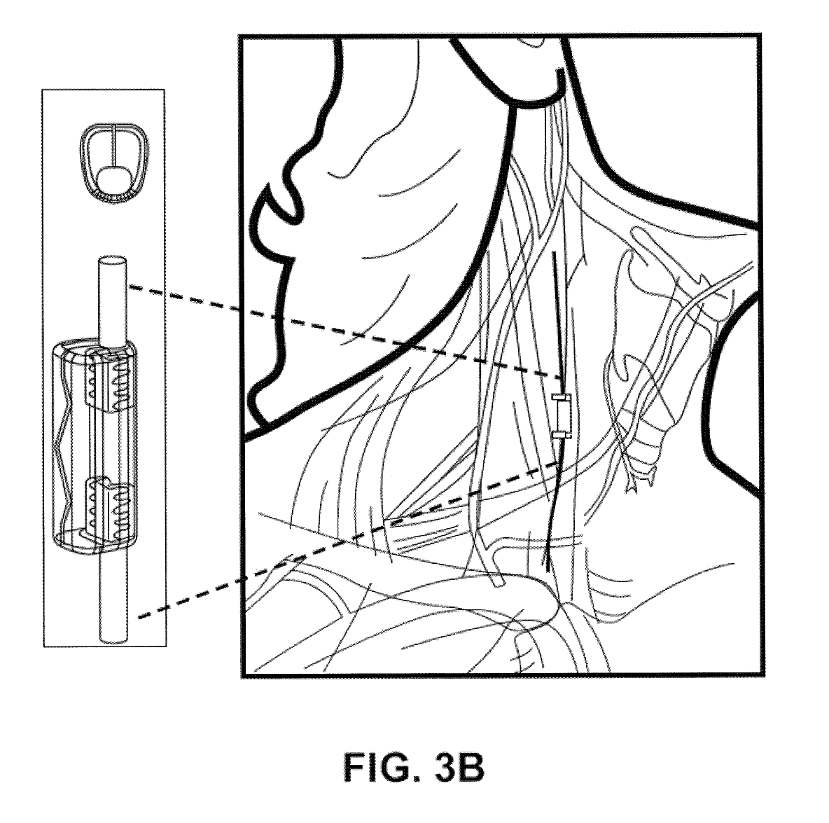

It some embodiments, an external programmer can be used as a controller that helps control and regulate the dose delivered by the system. In some embodiments, some or all of the functionality of the controller of the neck charger can be moved into the external programmer, allowing the external programmer to interface with and control the electrical stimulator. The external programmer can be a device that is worn on the body, such as the wrist, arm, neck, head, torso, leg, and/or ankle, that has a pair of straps for securing the device to a part of the body. In some embodiments, wearing the device on the wrist allows the user to more easily view and manipulate the external programmer. In some embodiments, the external programmer can be a smart watch or a wrist band device. For example, FIG. 7A illustrates a schematic of an embodiment of an external programmer 700. The external programmer 700 can have a processor 702 in communication with memory 704, a display 706 which can include a user interface such as a touchscreen and/or keyboard, a power source 708, and one or more communications features, such as near field communications (NFC) and/or wireless communications 712 (Wife, Bluetooth, etc.). Programming and instructions can be stored on the memory, and can be executed by the processor to perform the steps described herein. In some embodiments, the external programmer 700 and the electrical stimulator can use NFC to communicate with each other. NFC allows secure communications between the devices, and can help prevent unintentional changes to the stimulation protocol. In some embodiments, the NFC can be set to allow for communications within a predetermined range, such as 5, 4, 3, 2, 1, or 0.5 feet. In some embodiments, NFC can be used to communicate with a computing device, such as a smart phone. In addition, in some embodiments, a standard wireless communications protocol can also, or alternatively, be used. The wireless communication modalities can have a substantially longer range than NFC and can be used to communicate with a computing device, such as a computer or smart phone.

FIG. 7B illustrates how the external programmer 700, which can be a smart watch as shown, can be used to communicate with a remote computer 730 or computing device through the cloud 740 (or in some variations, as indicated by dashed lines, directly) to update the stimulation protocol delivered by the electrical stimulator 720, which can be a programmable microstimulator as shown and described herein. As shown in FIG. 7B and described above, the external programmer 700 can communicate with the electrical stimulator 720 through NFC or another wireless communication protocol. The external programmer 700 can then communicate, either wirelessly or through NFC, to a smart phone 750 or another computing device, such as a computer, which can be in communication with the remote computer 730 through the cloud 740. Optionally, the electrical stimulator 720 can communicate directly with the smart phone 750 through NFC. The smart phone 750 can connect to the internet and the cloud 740 through a wireless network or through a mobile telecommunication protocol such as a 3G or 4G network.

In other embodiments, the system can include just the external programmer 700 and the electrical stimulator 720, or the external programmer 700, the electrical stimulator 720, and the smart phone 750 or other computing device. The systems described herein can be used to remotely and/or locally adjust the stimulation protocol and/or parameters delivered by the electrical stimulator 720.

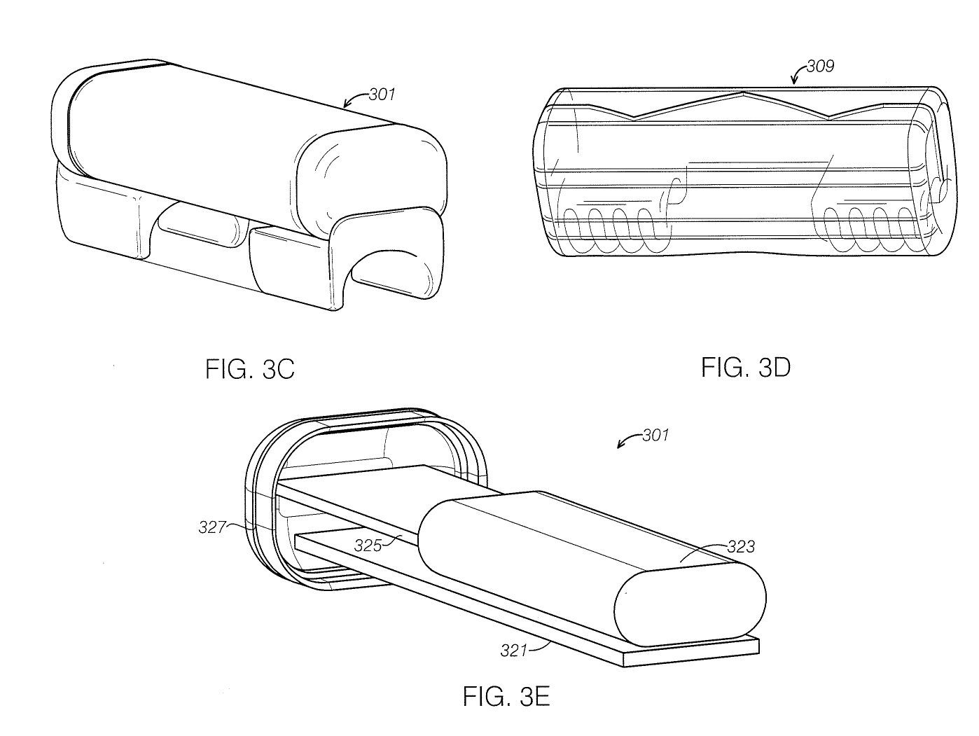

FIG. 8 is a flow chart illustrating one embodiment of how the stimulation protocol and/or parameters of the electrical stimulator can be updated. In step 800, the user can input and/or modify the stimulation protocol and/or parameters on the external programmer. In some embodiments, the external programmer has a touch screen interface and/or a keyboard and/or buttons that can be used to input information into the external programmer.

The external programmer can have one or more applications that facilitate the communication between the external programmer and the electrical stimulator. These applications can be updated and/or replaced, for example, through a computing device in communication with the cloud. In some embodiments, the application can allow the user to select a stimulation profile from a plurality of stimulation profiles. In some embodiments, the application can present a default stimulation profile.

In step 802, the external programmer can activate its NFC feature and/or its wireless communication protocol, such as Bluetooth and/or Wifi, that allow the external programmer to communicate with the electrical stimulator at close and moderate ranges, respectively.

In step 804, as the user moves the external programmer closer to the electrical stimulator, the system can determine, using NFC for example, whether the external programmer is within range of the stimulator. The system can keep checking until it determines that it is within range. Then, in step 806 it proceeds to update the stimulation protocol and/or stimulation parameters on the electrical stimulator using NFC and/or the wireless communication protocol.

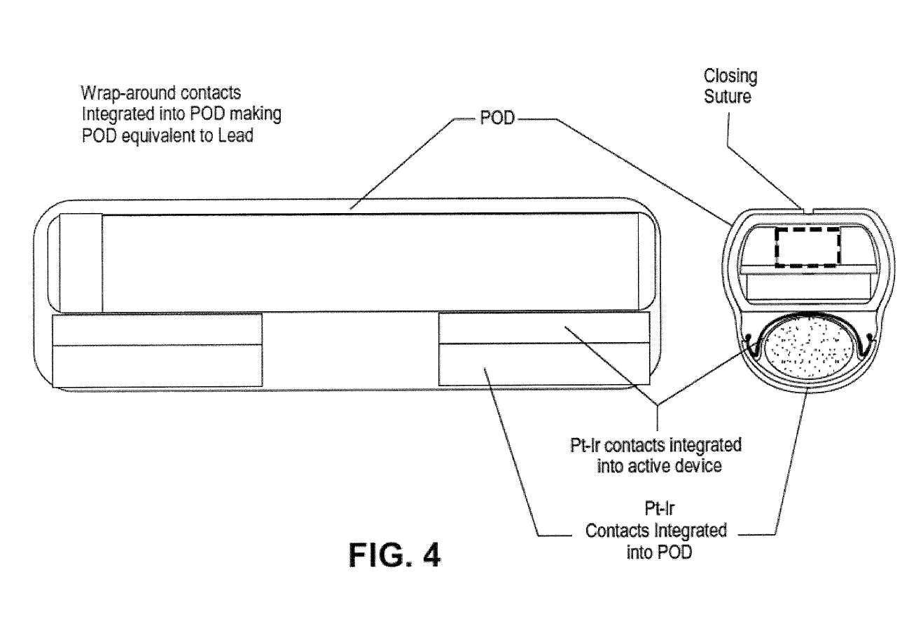

In some embodiments, as shown in FIG. 9, NFC can be used to pair the external programmer with the electrical stimulator, and then a wireless communications protocol, like Bluetooth, can be used to transmit data between the devices. Alternatively, Bluetooth can be used to pair the devices and for transmission of data. In step 900, the user can initiate pairing of the external programmer with the electrical stimulator. Next, in step 902 the external programmer can active NFC if close range pairing is desired, such as less than 5, 4, 3, 2, or 1 feet. Alternatively, in some embodiments, a wireless communication protocol, such as Bluetooth, can be activated instead for mid-range pairing where the devices can be separated by more than 5 feet. In step 904, the system can determine whether the external programmer and electrical stimulator are within pairing range. Once within range, the system proceeds to step 906 where the devices are paired together. Pairing can ensure that the external communicator only communicates with the proper electrical stimulator. It can also allow for close range pairing and longer range data communications one the proper link has been established, which may make it easier for the patient to update the electrical stimulator.

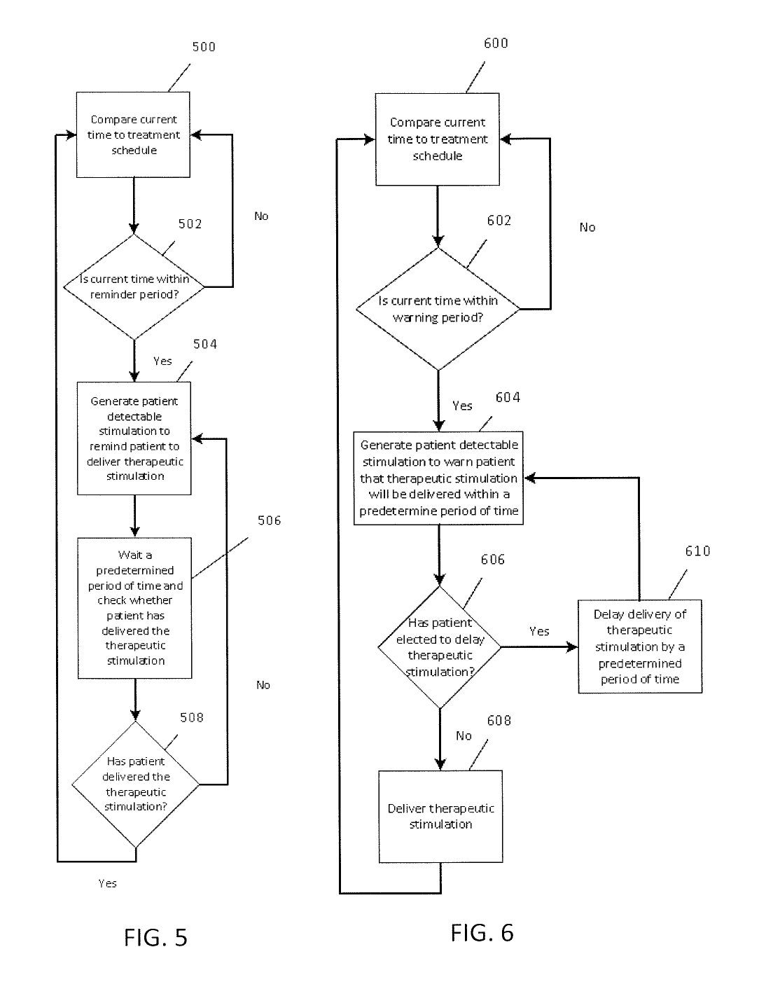

In some embodiments, as shown in FIG. 10, the stimulation protocol and/or parameters can be modified on a local computing device, such as a laptop, desktop, or tablet computer, which can then transmit the modified stimulation protocol to an external programmer. In step 1000, the user can input and/or modify the stimulation protocol and/or parameters on the local computing device. Then, in step 1002, the modified stimulation protocol and/or parameters can be transmitted to the external programmer, either wirelessly through Bluetooth or Wifi for example, or through a wired connection such as USB. Then, in step 1004, the external programmer can transmit the modified protocol and/or parameters to the electrical stimulator as described herein. For example, the external programmer can transmit data through NFC or a wireless communication protocol. Once the external programmer is brought within transmission range to the electrical stimulator, in step 1006, the external programmer can update the stimulation protocol and/or parameters using NFC or the wireless communications protocol.

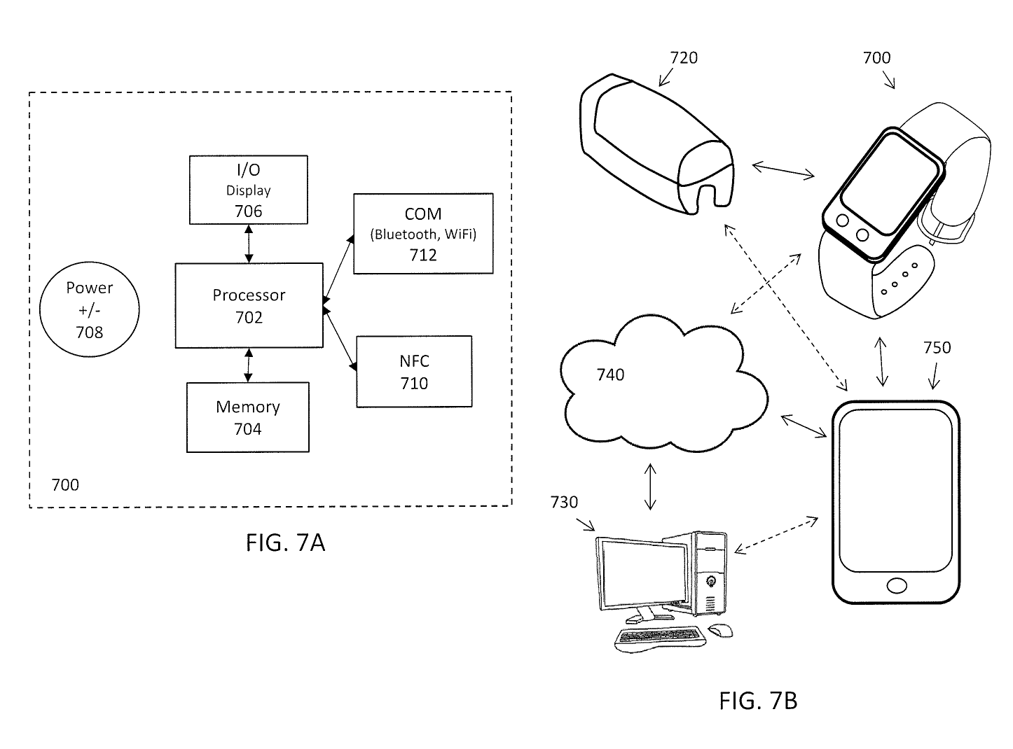

In some embodiments, as shown in FIG. 11, a remote computing device can be used to modify the stimulation protocol and/or parameters. The remote computing device can be used by a patient's health care provider to remotely update and/or modify the stimulation protocols and/or parameters used by the electrical stimulator. In step 1100, the remote operator can modify the stimulation protocol and/or parameters on a remote computing device with the appropriate software. In step 1102, the modified stimulation protocol and/or parameters can be transmitted to the cloud or server. In step 1104, the modified stimulation protocol and/or parameters can be transmitted to the patient's mobile device, such as a smart phone, or in some embodiments, directly to the external programmer. In step 1106, the modified stimulation protocol and/or parameters can be transmitted from the mobile device to the external programmer. In some embodiments, the patient can be given an alert on either the mobile phone and/or external programmer that an updated stimulation protocol and/or parameters has been received, allowing the patient to initiate the next step in updating the stimulation protocol and/or parameters. Once the patient initiates or acknowledges the update, in step 1108, the external programmer can check whether it is in range of the stimulator, as described above. Once the external programmer is in range with the electrical stimulator, the system and method can proceed to step 1110, where the electrical stimulator can be updated with the modified stimulation protocol and/or parameters using NFC or another wireless communication protocol.

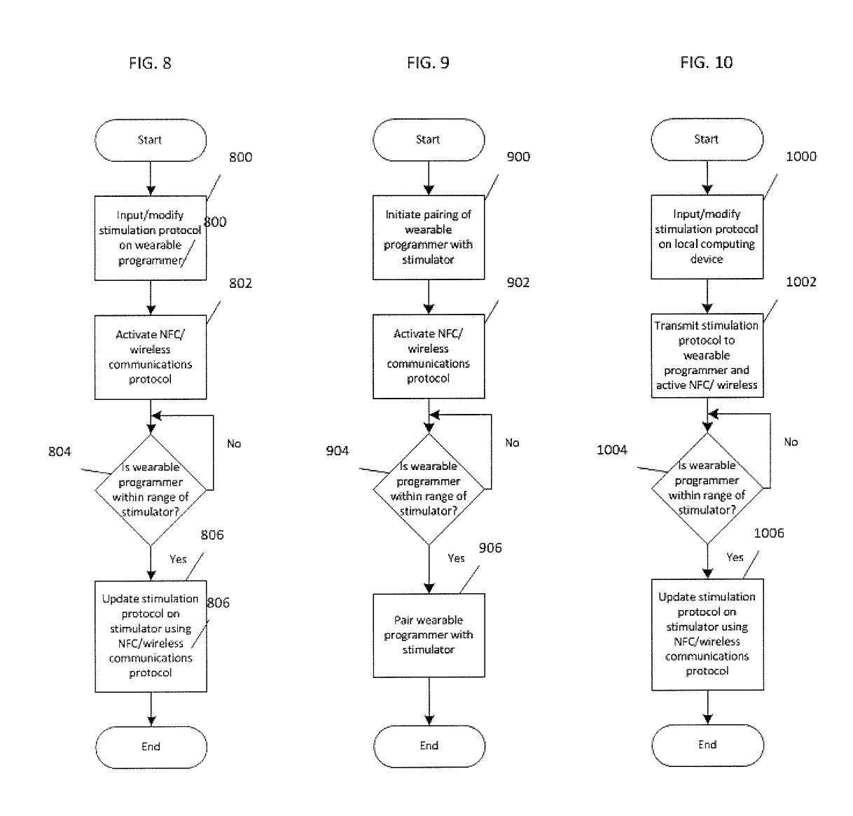

In some embodiments, the external programmer can deliver a reminder to the patient to deliver a stimulation, if the stimulator is in manual stimulation mode, or provide a warning to the patient that a stimulation is about to be delivered, if the stimulator is in automatic stimulation mode, as further described above. In order for the external programmer to deliver accurate reminders and/or warnings to the user, it is important to synchronize the stimulation protocol and/or schedule on the external programmer with the stimulation protocol and/or schedule on the electrical stimulator. FIG. 12 illustrates an embodiment of how the external programmer can be synchronized with the electrical stimulator. In step 1200, the external programmer can check, if within range, whether the stimulation protocol and/or schedule on the external programmer matches the stimulation protocol and/or schedule on the electrical stimulator. If the stimulation protocol and/or schedule is not the same, then in step 1202, the stimulation protocols and/or schedules can be synchronized. In some embodiments, the user can select which protocol or schedule to use for the synchronization. For example, both the external programmer and electrical stimulator can be synchronized to the stimulation protocol and/or schedule on the external programmer, thereby updating the stimulation protocol and/or schedule on the electrical stimulator. Alternatively, the stimulation protocol and/or schedule on the electrical stimulator can be used for synchronization if the user does not want to update the electrical stimulator. Once the devices are synchronized, in step 1204, the external programmer can provide warnings or reminders to the patient a predetermined time before a scheduled stimulation, as further described above. The warnings and reminders can be visual (i.e., flashing light or pop up message), auditory (i.e., beep or message), and or tactile (i.e., vibration).

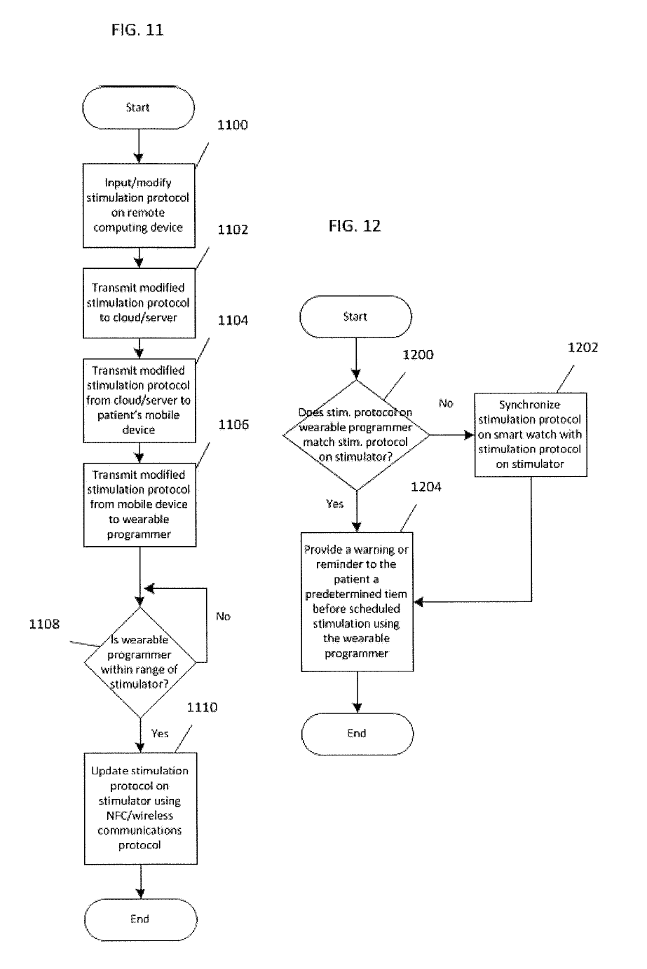

In some embodiments, as shown in FIG. 13, the external programmer can be used to deliver manual stimulation through the electrical stimulator. In step 1300, the external programmer can check whether manual stimulation has been enabled and/or prescribed. If manual stimulation has not been enabled or prescribed, the user may be prevented from delivering manual stimulations until manual stimulation is activated on the external programmer. In some embodiments, activation of manual stimulation requires input or confirmation from the patient's health care provider. For example, the health care provider may be required to enter a code to enable manual activation. In some embodiments, the code may be entered on a remote computer and then transmitted to the external programmer through the cloud or network. If manual stimulation is enabled, in step 1302, the patient can administer a manual stimulation using the external programmer. In some embodiments, the patient can push a button to deliver the stimulation. In other embodiments, the external programmer can have a touch screen which the patient can touch to deliver a manual stimulation. In some embodiments, the user must further confirm the delivery of a manual stimulation with another button push or another touch of the screen. Once the manual stimulation is delivered, the external programmer can log the delivery of the manual stimulation to the patient, as shown in step 1304. In some embodiments, the log can be automatically and/or manually uploaded to the cloud or a server, which can then be accessed by the patient's health care provider to monitor the patient's compliance with the stimulation protocol and schedule.

In some embodiments, the external programmer can store and present to the user a plurality of stimulation profiles that the user can select. In some embodiments, the external programmer can allow the user to modify a select set of stimulation parameters, such as stimulation amplitude, duration, and/or frequency of stimulations (i.e. once daily, twice daily, etc.). In some embodiments, the external stimulation can provide warnings and/or reminders to the patient of upcoming stimulations, and can also allow the patient to delay stimulation, as described above. In some embodiments, the external programmer can display to the user to status of the electrical stimulator, such as the power or charge remaining in the stimulator, and whether the electrical stimulation should be recharged. In some embodiments, the external programmer can display to the user the stimulation schedule and/or parameters.

In some embodiments, the external programmer can be waterproof so that the user can wear the device while taking a shower or taking a bath. In some embodiments, the external programmer can have a scratch resistant screen and be impact resistant. In some embodiments, the external programmer can have a typical smart watch or wrist band device (i.e. Fitbit) form factor to make it difficult for a casual observer to determine that the patient is wearing a medical device on his/her wrist.

In some embodiments, the external programmer can be in communication with one or more sensors that allow the external programmer to be used in a closed loop stimulation system with the electrical stimulator. For example, the external programmer can be in communication with an EEG device and/or EEG leads that measures P300 and/or the activation of the nucleus basalis and/or the locus coeruleus, as further described in U.S. Provisional Application No. 62/068,473, which is herein incorporated by reference in its entirety, and can modulate the stimulation protocol and/or parameters based on these EEG readings. Another embodiment of a closed loop system can use an electrode, which may be included in the electrical stimulator, that can be used to detect vagus nerve activity, as further described in International Patent Application No. PCT/US2014/033690, which is herein incorporated by reference in its entirety. The external programmer can be in communication with the electrode and can determine vagus nerve activity, by for example counting neural spikes in the vagus nerve, and can modulate the stimulation protocol and/or parameters based on the detected vagus nerve activity.

### Control of Patient Information

In any of the apparatuses (systems and devices) described herein, patient information, and particularly information about the status of the implant and patient interactions (and in some cases physiological responses to the implant) may be stored in a stable memory on the system and cannot be removed, even if transferred (uplinked/uploaded) from the implant, e.g., through the programmer (e.g., in some variations, the “energizer”). Thus, in some variations the implant may include a memory that is configured to be written by the controller in the implant with any of the status information described herein (e.g., log information described below, such as programmed dosing schedule, actual doses delivered, error codes, patient commands, electrode resistance/impedance information, etc.); the memory may be indelible, so that once it is written, it cannot be modified (e.g., deleted, changed, etc.). Thus, the information may be stable even if the device loses power or suffers from a failure that would otherwise disable the device. The memory may therefore serve as a “black box” (e.g., similar to an airplane flight recorder), allowing the device to be removed from the patient so that the memory can be examined to determine operational parameters and/or patient status while it was running, and may include error codes (as well as times that the error code was entered).

This memory may also serve as a patient compliance record, which records every dose delivered, thereby allowing confirmation that a patient is complying with the prescribed treatment.

Thus, any of the implants (e.g., micro regulator or micro-stimulator) described herein may keep an indelible record of all relevant status information (e.g., programmed dosing schedule, actual doses delivered, error codes, patient commands, electrode resistance/impedance information, etc., each of which may be time-stamped) the records (ONLY on the micro-regulator) this logged information (“log”) is kept with the patient as a medical record. This may ensure both patient safety as well as patient medical record security.

In some variations, the implant may be configured with security features that prevent the stored log information from being transmitted (e.g., wirelessly transmitted) from the implant without the proper security being entered. In addition, the implant's controller (microcontroller) may be configured to encrypt the information stored and/or transmitted so that it can only be decoded by a user having the encryption key.

In any of these variations, the apparatus may be configured so that the log information in the indelible memory can only be transmitted during charging of the device, e.g., when connected to a programmer/energizer). In general, any of these apparatuses may be configured so that telemetry is disengaged (e.g., wireless communication) unless the implant is receiving power from the programmer/energizer. For example, the apparatus may be configured so that the log information in the indelible memory may only be transmitted when telemetry is activated and the device is receiving power and/or instructions from the energizer/programmer. The telemetry portion of the implant may be off when not charging from an energizer/programmer. Further, the connection to the energizer/programmer may require validation by the implant. Thus, in some variations, in order for the apparatus to transmit any of the log information from the implant, the patient must essentially be physically present, and connected to an energizer/programmer.

In general, implant may store in the indelible memory (and note that additional memory may be included as part of the implant, including rewritable memory) any status information, including specifically: sensor readings (e.g., electrode impedance, internal device temperature, etc.), internal diagnostics testing results, error codes (e.g., emergency shut downs, low power indicators, digital fault codes), patient commands (stop override commands, etc.), prescription/stimulation instructions (e.g., current amplitude, pulse width, burst duration, burst frequency, etc.), physician tests (e.g., testing stimulation protocols), and the like. Any of this data may also be time stamped by the microprocessor (which may include clock circuitry) so that the data is entered with an indication of the relative (to the implant) or absolute (to the world outside the implant, e.g., GMT) time. Additional information that may be stored includes: sensor readings, such as patient temperature (optional), etc. In general, any of this information may be included or not included, though it may be particularly helpful to include information about the dosing instructions and the actual delivered doses (e.g., time of delivery, amplitude/voltage delivered, frequency within bursts, burst frequency, number of bursts, off-time between doses, etc.).

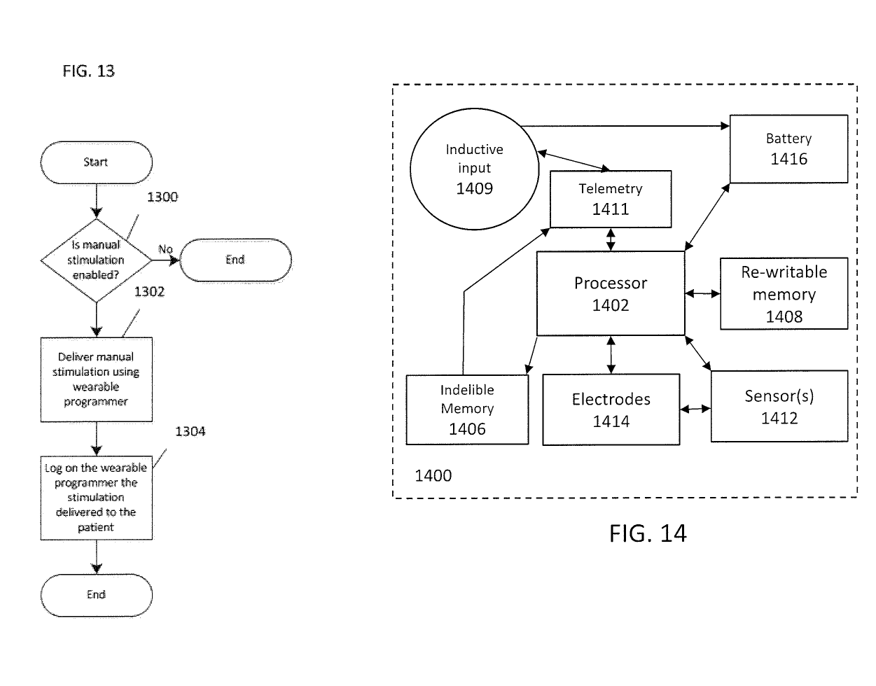

For example, FIG. 14 schematically illustrates components of an implant 1400 as described herein, including the microcontroller processor 1402 that controls the application of stimulation on to the electrodes 1414. One or more sensors 1412 (e.g., temperature sensors, impedance sensors, etc.) may be included, and may communicate directly with the electrodes 1414 and/or the processor 1402. A transient (e.g., read/write) memory 1408 may also be included, either separate from or integrated into the processor 1402. An indelible memory unit 1406 may also be included and connected to the processor for receiving (writing) any of the status (log) information described herein in a permanent or near-permanent form. The apparatus may also include an induction coil 1409 for receiving power (and near-field communication) as described above from an external energizer (programmer), which may also provide power to the telemetry circuitry 1411; in some variations, the telemetry is only “on” when receiving power from the inductive input. Signals may be transmitted via the inductive input, for example, by modifying the load (e.g., inductance) of the input 1409 which can be detected by the energizer/programmer. Similarly, information (e.g., dosing information) transmitted to the implant via the telemetry may be encoded in the charging signal. Thus, no separate communication means (wireless, e.g., Bluetooth, WiFi, etc.) is necessary. As discussed above, the telemetry circuit may be inactive unless it is receiving power from the inductive input 1409. The battery may be charged by the inductive input, and battery power may be monitored by the processor 1402.

### Clock Variations

Embodiments of the present invention are directed towards systems and methods for performing calibration of a first clocking mechanism, where the calibration occurs periodically or based upon some event or signal being detected and through use of a second, more accurate clocking mechanism. It is desirable to maintain an accurate clock in the implant in order to reliably deliver programmed stimulations at the scheduled times.

In implantable devices, and many other electrical devices in general, there is great demand for having systems with lower power consumption as well as lower cost. Lower power expenditure may be achieved through having a process that does not draw as much power, but often this is at the expense of having less accurate outputs. In the case with a clocking system, the use of a less accurate clock signal may lead to lower power consumption compared to a more accurate clocking mechanism, but a less accurate clock having lower power consumption may result in providing output at imprecise or unpredictable times.

One way to compensate for having a systems clocking mechanism that is a less accurate clocking mechanism that will be periodically calibrated with a more accurate clocking system. Disclosed herein is a first or central clocking mechanism that uses a semiconductor junction to generate a reference voltage that in turn charges an RC circuit to produce a time reference. Because these voltage references have significant variations due to integrated circuit characteristics and parameters and temperature, they tend to be less accurate.

To compensate for the lack in accuracy of the first clocking mechanism, a second more accurate clocking mechanism can be employed. The second, more accurate clocking mechanism may be used to periodically recalibrate the first clocking mechanism.

More accurate clocking mechanism include real time clocks. Real time clocks are a type of computer clock in the form of integrated circuits. Most real time clocks use a piezoelectric crystal oscillator, where the oscillator frequency is 32.768 kHz, the same frequency as in quartz clocks and watches.

In one non-limiting example, a time reference clocking module error in the RC circuit may be measured over fixed intervals or based on a change in a pre-determined parameter (e.g. voltage or current). Deviations may be measured against a more accurate real time crystal oscillator clocking mechanism. Based on the measured deviation and time elapsed since the last calibration, the amount of time deviation in the time reference clocking module may be calculated and corrected. Correction of any time deviation may be occur through correcting the central clocking module. Alternatively, the central clocking module may be temporarily replaced with the more accurate real time crystal oscillator clocking mechanism to bring the central clocking module back to a correct value.

In another non-limiting example, the implantable device will run the central clocking mechanism continuously while a second, more accurate clocking mechanism remains in an OFF or standby mode. Upon the occurrence of a pre-determine event or time interval, the second, more accurate clocking mechanism may enter an active mode and re-calibrate the central clocking mechanism. Upon completion of the calibration routine, the second, more accurate clocking mechanism will again revert to an OFF or standby mode until the next calibration is triggered.

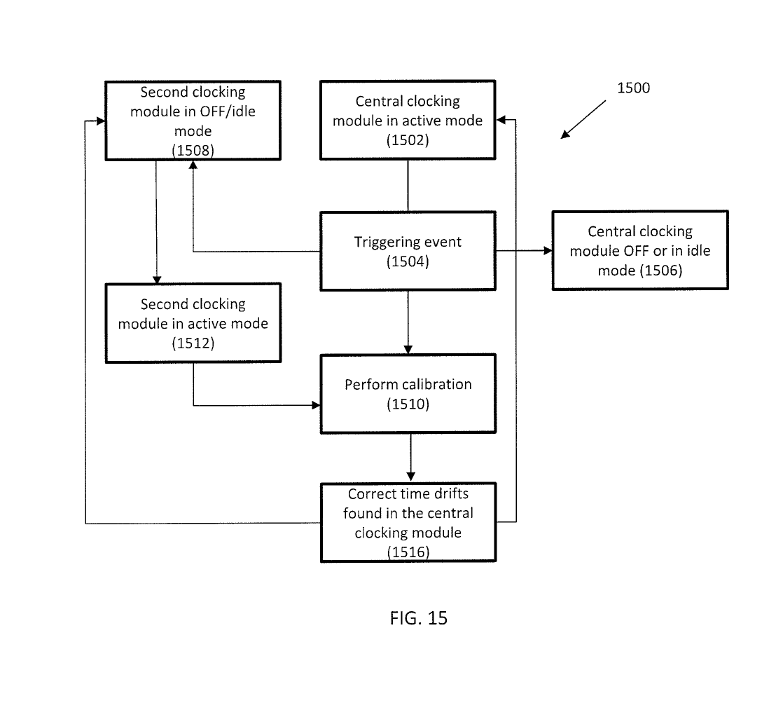

FIG. 15 shows a flowchart for visualizing the steps of implementing a clocking calibration routine 1500. Presumably the clocking system for the implantable device, such as a neurostimulator, will be activated once the device is implanted in the patient. At 1502, the systems clocking mechanism is running. At this point, the second, more accurate clocking mechanism is in a sleep or OFF mode (1508). At some point in time later, an even triggers a signal being sent to the second clocking mechanism (1504). The trigger may correspond to the beginning of a new cycle in the implantable device. In the case of an implantable neurostimulator, the trigger may be associated with the beginning of a stimulation session or a combination of features of the stimulation session (e.g. a time interval after the start of the stimulation session). A trigger for calibration may also be a circuit parameter that has exceeded or dropped below a threshold value. Once the trigger event has occurred (1504), a signal is sent to the second clocking module to turn from the OFF or standby mode to an active mode (1512). With the second clocking module in an active state, it will initiate the calibration routine (1510). Once the calibration routine (1510) has been performed, any deviation determined from running the calibration routine (1510) may be corrected in the following step (1514). Once the deviation has been corrected, a second signal may be sent to the second clocking module to return to an OFF or standby mode. In the final step, a third signal may be sent to the central clocking module to switch it from an OFF or standby mode to an active mode. These steps may be repeated based on a condition being satisfied, an event occurring, or a pre-determined period of time. Also, it may be possible to delay calibration to sometime past the triggering event.

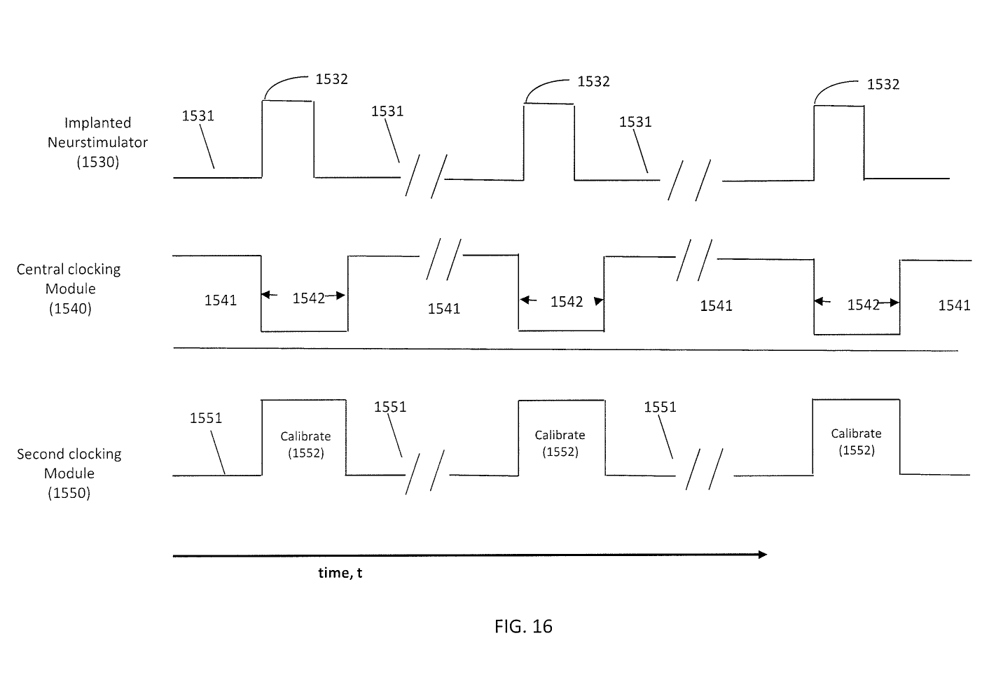

Turning to FIG. 16, a sample calibration routine based on some feature or characteristic of the implanted device output is shown. In the case of an implantable neurostimulation device, calibration of the central clocking system may be tied to when a stimulation session begins. In this scenario, a sensor may be incorporated to sense when a current or voltage has increased above a certain value and that a calibration routine should be initiated immediately or after a set amount of time. To better visualize each component status, FIG. 16 shows stacked signals in order from top to bottom: a series of neurostimulation outputs for a neurostimulator 1530, the functional state of the first or central clocking module 1540, and the functional state of the second clocking module 1550 that is able to perform the calibration routine. The horizontal axis from left to right indicates the passage of time and may be in units of minutes, hours, days, weeks, months, and so forth.

As the diagram arbitrarily shows a snapshot of the output of an implanted device. Initially, when the neurostimulation device is in an idle state (1531), the central clocking module 1540 is in an active mode 1541 and the second clocking module 1550 is in an OFF or standby mode (1551). The central clocking module 1540 will then continue to run for some period 1542 until a neurostimulation session begins (1532), at that point, signals are set to the both the central clocking module 1540 and the second clocking module 1550 when the neurostimulation output surpasses a certain threshold value. Upon reaching this state, the central clocking module will drop to an idle or OFF state 1542 while the second clocking module 1550 will switch from its OFF or standby mode 1551 to an active mode 1552, where it will run a calibration routine 1553 either immediately or at a preset time in the future. Upon completion of the calibration routine 1553, a signal is sent to the central clocking module to coordinate switching it from the standby mode 1542 back to an active mode 1541 and for the second clocking module to return from an active mode 1552 to an OFF or standby mode 1551 in a coordinated fashion. These steps will repeat based on some feature of the stimulating output from the implanted device. In some other variations, the calibration routine may be tied to some other feature of the stimulating output and not necessarily correspond to the beginning of the stimulation output.

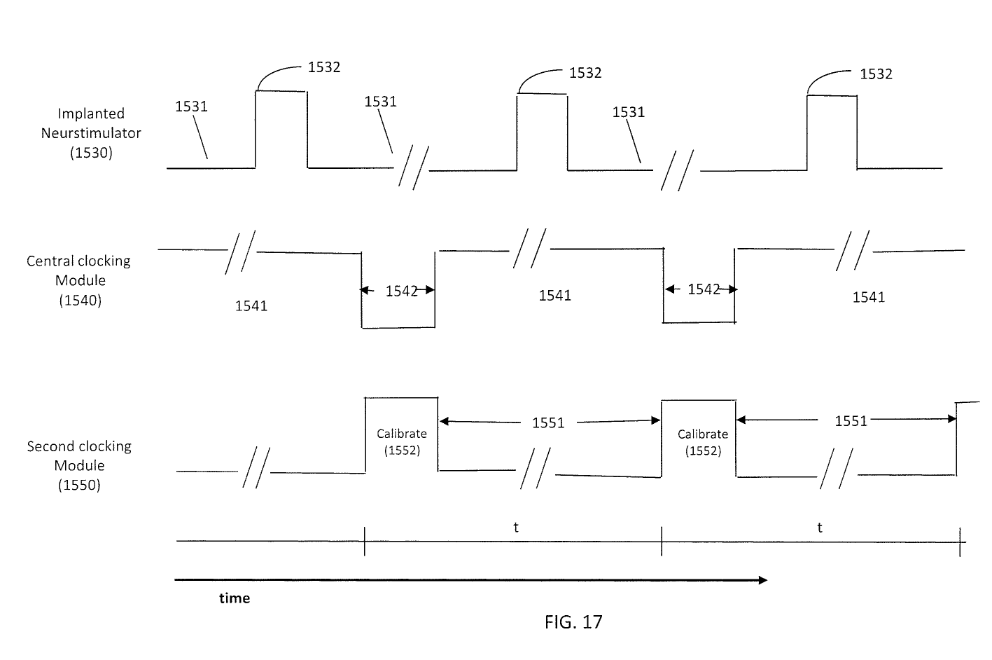

FIG. 17 shows an alternative initiation of calibration routines in a system where a secondary, more accurate clocking module is used to calibrate and correct any deviations experienced by a less accurate central clocking module. In this arrangement, the implanted device output will provide output periodically, where the time periods may be the same or different and may be set by the doctor or other user. A calibration routine may occur that aligns with a given time period t, that repeats. As the diagram shows, during the evolution of time period t, the central clocking module is in an active state 1541, while the second clocking module is in an OFF or inactive state 1551. At the end of the time period t, the central clocking module will switch to an idle or OFF mode 1542 while the second clocking module will turn to an active mode 1552 to calibrate the less accurate central clocking module 1540 and adjust for any deviations that is measured. Upon completion of the calibration routine the second clocking module 1550 will return to an OFF or standby mode 1551 while the central clocking module 1540 will return to an active mode 1541. These steps will repeat based on a pre-defined time interval. In some examples the time period will be the same, but in other examples the time period may be different or may be based on some algorithm or known relation between the length of time and the amount of deviation expected.

The second clocking module may be linked to the calibration module that performs the actual calibration routine. The calibration module may be integrated into the circuitry of the implanted device. The systems clocking module is able to provide a central clocking signal that serves as a clock source.

In some other examples, the systems clocking module is configured to provide a tick signal that acts as a time keeper. Periods between device outputs may be defined by the number of tick counts. While the tick counts accuracy is based upon characteristics of the circuit parameters, and may be not be as accurate as some other timing keeping mode, certain methods may be implemented to accommodate any inaccuracies. For example, tick counts may be tied to the calibration module, which can be used to determine the duration of intervals between successive calibration routines. The start of a calibration routine is initialed by a signal which is configured to count the ticks from the central clocking module. The ticks may be counted until the calibration routine is complete and through a period where the central clocking module is keeping time. Tick counts may restart based upon the start of a new calibration routine. Every time the calibration routine is run, any deviations resulting from the tick counts may be corrected. In the example of an implantable neurostimulation device that has wireless recharging capabilities, the tick counts may be adjusted for accuracy using a more accurate time keeper located within the wireless transmitter unit. Thus, whenever the implanted neurostimulation device is being recharged, the tick counts may be matched with the more time keeping module within the wireless transmitter unit and any deviations may be corrected. The benefit of having a tick counting type time-keeping module is that a patient may move to different time zones without having to modify potentially salient circadian components of the stimulation output.

As alluded to above, the implanted device circuitry or controller may also be configured to detect a trigger or event that will commence a calibration routine. The trigger may be an increase in a threshold voltage or current value. The trigger may also be a combination or a pattern of changes in the voltage or current value in more complex arrangement of stimulating outputs.

The implantable device may also be configured to provide a series of signals that will coordinate the switching of the central clocking module from an active mode to an OFF or standby mode, while signals are also sent for switching the second clocking module from an OFF mode to an active mode for the calibration routine.

The implantable device may also include programs or algorithms that will be able to correct for any time drift that may be detected after the calibration routine is completed. In another variation, the step of calibrating the central clocking module and accounting for any deviation may be performed in one step.

In some non-limiting variations of the clocking calibration systems and methods, the implantable neurostimulation device may be able to retain information on the calibration results such as the amount of drift that the central clocking module has experienced since the previous calibration routine. This information may be sent wirelessly to a telecommunication device or may be sent to the wireless transmitter module during recharging events.

It should be noted that because the clocking system described herein is directed to use within an implantable device, there is minimal temperature variations that may cause further drifts in the clocking system. Because the implant is in a temperature stable environment, there may be no need for temperature compensation. The circuit's wafer to wafer and die to die variations may be calibrated to a fixed temperature and scaled to 37° C. during manufacturing of the implantable device, may be calibrated during the programming of the implantable device, or during the wireless charging process.

In yet other variations, the calibration routine and subsequent correction steps may be in response to a received voltage or current signal from a sensor via some data communication link, and compares the received voltage or current signal against a set of pre-programmed or learned variables and values to determine if the central clocking module needs to be recalibrated. While the calibration routine may occur at any time, it may be beneficial to run the calibration routine when there is no stimulating output being provided. This would prevent overtaxing the overall circuitry of the implanted device.

It should also be noted that what is shown in the figures and disclosed above only exhibit a few possibilities for which a second, more accurate clocking mechanism may be able to calibrate and correct any drifts in the time of the less accurate central clocking module. The examples shown are merely to illustrate and instruct and are not intended to be limiting in any way. Thus, there are numerous calibration sequences that may be implemented using the basic concepts laid out above.

### Remote Control

In some embodiments, the second, more accurate clock can be located within an external device, such as the external programmer described herein, or a separate external device that may be worn, attached to clothing, or carried by the user. Devices that can be worn include the energizer/charger described herein or a device worn over the wrist or around the neck.

In some embodiments, the energizer can be used to calibrate the internal clock in the implanted device when the energizer is used to charge the implant. The energizer can have a more accurate clock, such crystal oscillator based clock. However, in some embodiments, the implant may only need to be charged weekly, monthly, quarterly, semi-annually, or annually, while it may be desirable to calibrate the internal clock of the implant on a more frequent basis based on the level of accuracy of the clock. For example, in some embodiments, the internal clock of the implant may need to be calibrated daily or at some other relatively frequent basis in order to maintain the clock within a predetermined level of accuracy, such as within 30, 20, 10, 5, 4, 3, 2, 1 minutes, while the implant only needs to be charged about every 4, 5, 6, 7, or 8 months. Instead of using the energizer to calibrate the clock on a daily basis, a more convenient external programmer or another external device can be used instead.

The external programmer or another external device can function as a remote control device that can be used instead of or along with the energizer to calibrate the internal clock of the implant and control other aspects of the implant. With the energizer, the power signal from the energizer can be used to both charge the implant and to communicate with the implant in order to update the clock and provide additional instructions to the implant or retrieve data from the implant. However, with an external programmer or other external device, it would be desirable to be able to communicate with the implant without the need to use a high power signal.

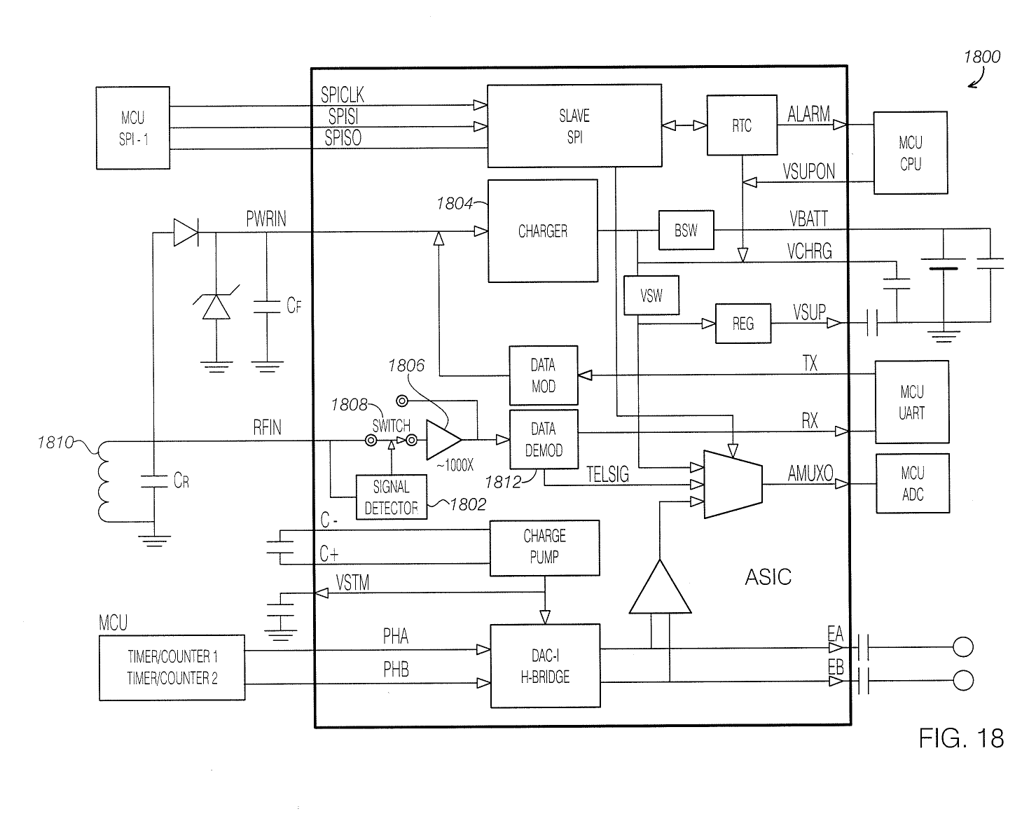

For example, in some embodiments as shown in FIG. 18, the implant 1800, which can be a microstimulator, can have a low power signal detector 1802 that can be internally powered, by a power source such as a battery 1804 for example, in order to detect a low power signal from the external programmer or other external device that can be used for communications. The low power signal detector 1802 can be powered continuously or intermittently in order to conserve power. Upon detection of a low power signal, an amplifier 1806 can be used to amplify the low power signal before it is processed. The amplifier can amplify the signal by about 100×, 1000×, or 10000×; or about 2, 3, or 4 orders of magnitude. A switch 1808 can be used bypass the low power signal detection circuit until the low power signal is detected. The low power signal detection circuit can be placed between the antenna 1810 and the signal processing components, such as the data demodulator 1812. The low power signal detector 1802 can be tuned to a predetermined frequency that matches the frequency of the low power signal sent by the external programmer or other external device. For example, the low power signal detector 1802 and amplifier 1806 can selectively amplify signals with a frequency of 131 kHz, which is used by the low power signal, and have a sensitivity to detect 0.1 to 1 mV signals. The amplified signal can be passed to the data demodulator 1812 for further processing.

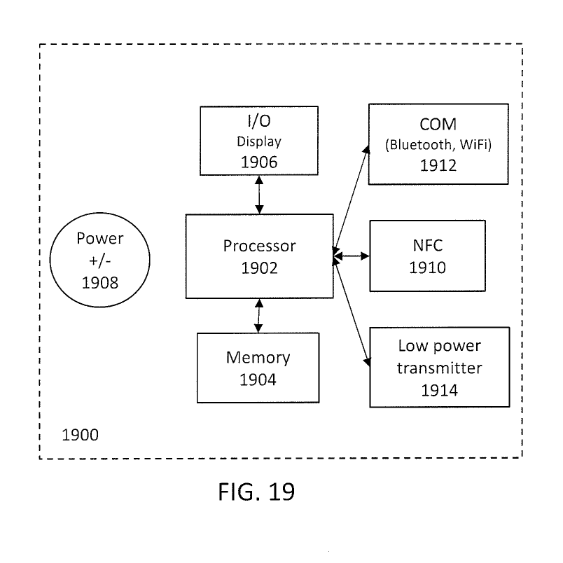

As shown in FIG. 19, the external programmer or other external device 1900 can have a low power transmitter 1902 that generates the low power signal. The external programmer or other external device 1900 can be a device that is worn on the body, such as the wrist, arm, neck, head, torso, leg, and/or ankle, that has a pair of straps for securing the device to a part of the body, or can be fastened to clothing or be carried. The external programmer or other external device 1900 can have a processor 1902 in communication with memory 1904, a display 1906 which can include a user interface such as a touchscreen and/or keyboard, a power source 1908, and one or more communications features, such as near field communications (NFC) 1910 and/or wireless communications 1912 (Wifi, Bluetooth, etc.) and/or a low power signal transmitter 1914. Programming and instructions can be stored on the memory, and can be executed by the processor to perform the steps described herein.

The low power signal can be used to communicate with the implant and can be used to calibrate the clock, send commands and instructions to the implant, and/or update the implant software and parameters. For example, the low power signal can pause and restart stimulation, increase dosing, enable manual dosing, check battery level, perform diagnostic functions, and allow networking of a plurality of devices such as one or more sensors with one or more implants. This allows the external programmer or other external device to function as a remote control for the implant.

In some embodiments, an auto trimming oscillator, for example as described in U.S. Pat. No. 7,994,866 to Fievet et al., can be used to adjust the clock frequency on the implant to match the clock frequency in the external programmer or other external device.

In some embodiments, the implant generates a weak signal that may be difficult for the external programmer or other external device to detect unless the external programmer or other external device is brought within a predetermined distance from the implant, such as within about 12, 11, 10, 9, 8, 7, 6, 5, or 4 inches. In some embodiments, the external programmer or other external device can generate an alert, such as an audio, visual, and/or vibrotactile alert, when a signal is sent to the implant and/or received from the implant. In some embodiments, the external programmer or other external device can prompt the user with an audio, visual, and/or vibrotactile cue to move the external programmer or other external device to within a predetermined distance from the implant in order to facilitate bidirectional communication and to receive data from the implant, which is important for receiving confirmations from the implant and performing diagnostic functions.

In some embodiments, an intermediary device can be used to facilitate bidirectional communication with the external programmer or other external device and the implant. The intermediary device can be worn within a predetermined distance from the implant, which allows the intermediary device to receive a relatively weak signal from the implant and then transmit that signal to the external programmer or other external device. The intermediary device can also be used to receive signals from the external programmer or other external device and then transmit these signals to the implant. For an implant place in the patient's neck, the intermediary device can be worn around the neck, as a necklace for example, or be fastened to the patient's clothing, such as the collar or lapel.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An implantable neurostimulator system using an implantable low-power clock, the system comprising:
an implantable neurostimulator having a first clock configured to keep time within the implantable neurostimulator, a rechargeable battery, a pair of electrodes configured to be placed in electrical communication partially around a nerve, and a processor configured to deliver electrical stimulation from the electrodes according to a set of stimulation parameters; and
an external clock synchronizing tool, the external synchronizing tool configured comprising a second clock having more accurate time-keeping capabilities than the first clock and control circuitry configured to wirelessly communicate with the implantable neurostimulator and transmit a calibration signal,
wherein the processor is further configured to calibrate the first clock based on the calibration signal.

2. The system of claim 1, wherein the external clock synchronizing tool and the implantable neurostimulator are configured to communicate with each other through near field communication.

3. The system of claim 1, wherein the external clock synchronizing tool is a configured to be worn around the patient's wrist.

4. The system of claim 1, wherein the external clock synchronizing tool is a smart watch.

5. The system of claim 1, wherein the external clock synchronizing tool is configured to be worn on the patient's neck or torso.

6. The system of claim 1, wherein the external clock synchronizing tool is configured to interface with a local computing device.

7. The system of claim 1, wherein the first clock comprises an RC oscillator.

8. The system of claim 1, wherein the first clock varies by greater than 5 minutes per day.

9. The system of claim 1, wherein the external clock synchronizing tool is configured to periodically transmit the calibration signal.

10. A method of periodically calibrating a clock within an implantable neurostimulator device, the method comprising:
keeping time in the implantable neurostimulator device using a first clock located within the implantable neurostimulator device, wherein the first clock runs continuously;
wirelessly transmitting a calibration signal from an external clock synchronizing tool to the implantable neurostimulator device, wherein the external clock synchronization tool includes a second clock having more accurate time-keeping capabilities than the first clock; and
calibrating the first clock in the implantable neurostimulation device based on the calibrated signal received by the implantable neurostimulator device from the external clock synchronization tool.

11. The method of claim 10, wherein wirelessly transmitting a calibration signal comprises transmitting by a near field communication protocol.

12. The method of claim 10, further comprising applying neurostimulation to a patient's vagus nerve at a predetermined schedule based on output from the first clock.

13. The method of claim 10, further wherein wirelessly transmitting a calibration signal comprises transmitting the calibration signal when the external clock synchronizing tool is worn on a subject's neck or torso.

14. The method of claim 10, further comprising transmitting an on-demand stimulation request from the external clock synchronizing tool and receiving the on-demand stimulation request in the implantable neurostimulator device, wherein receipt of the on-demand stimulation request causes the implantable neurostimulator device to deliver electrical stimulation from a pair of electrodes to a subject's vagus nerve.

15. The method of claim 10, further comprising transmitting a hold stimulation request from the external clock synchronizing tool and receiving the hold stimulation request in the implantable neurostimulator device, wherein receipt of the hold stimulation request causes the implantable neurostimulator device to suspend electrical stimulation from a pair of electrodes to a subject's vagus nerve.

16. The method of claim 10, further comprising pairing the external clock synchronizing tool with the implantable neurostimulator device before wirelessly transmitting the calibration signal.

17. A method of calibrating a clock within an implanted neurostimulator device, the method comprising:
keeping time in the implanted neurostimulator device using a first clock located within the implanted neurostimulator device, wherein the first clock runs continuously;
periodically wirelessly transmitting a calibration signal from an external clock synchronizing tool to the implanted neurostimulator device, wherein the external clock synchronization tool includes a second clock having more accurate time-keeping capabilities than the first clock; and
calibrating the first clock in the implanted neurostimulation device based on the calibrated signal received by the implanted neurostimulator device from the external clock synchronization tool.
